SEQUENCE LISTING (109 sequences)

SEQ ID NO: 1 - protein, 726 aa, Homo sapiens
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALSADGKGSKGSEEDVVSKNQGDSARKQPGSSTSSSSHLAKPSSSRLCDTSSARQESTSKADLNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWDCCSTSYAQNLDFESSEGNTIANSVGEISSKLSEKSGLCLSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSSASPSKATKS

SEQ ID NO: 2 - protein, 726 aa, Pan troglodytes
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICKTALSADGKGSKGSEEDVASKNQGDSARKQPGSSTSSSSHLAKPSSSRLCDTSSARQESTSKADLNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWDCCSTSYAQNLDFESSEGNTIANSVGEISSKLSEKSGLCLSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSNASPSKATKS

SEQ ID NO: 3 - protein, 726 aa, Nomascus leucogenys
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALSADGKGSKGSEEDVASKNQGDSARKQPGSSTSSSSHLAKPSSSRLCDTSSARQESTSKAELNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWDCCSTSYAQNLDFESSEGNTIANSVGEISSKLSEKSGSCVSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSNASPSKATKS

SEQ ID NO: 4 - protein, 726 aa, Gorilla gorilla
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLGLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALSADGKGSKGSEEDVASKNQGDSARKQPGSSTSSSSHLAKPSSSRLCDTSSARQESTSKAELNCSKNKDLYQEQVEVMLDVTDTSMDTYLEREWGNKPSDCVPYKDEELYDLPAPCTPLSLSCLQLSTPENRESSVVQAGGSKKHSNHFRKLVFDDFCDSSNVSNKDSSEDDISRSENEKKSECFSSPKTGFWDCCSTSYAQNLDFESSEGNTIANSVGEISSKLSEKSGSCLSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQAEFSQGILLSSSHRLFEDQRFGSSLFKMSSEMYSLHNHLQSPWSASFVPEKRNKNVNQSTKRKIQSSLSNASPSKATKS

SEQ ID NO: 5 - protein, 726 aa, Pongo abelii
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETSRLKKALERSDKYIEELESQVAQLKNSSEEKEAMNSICQTALPADGKGSKGSEEDVASKNQGDSARKQPSSSTSSSSHLAKPSSSRLCDTSSARQESTSKAELNCSKNKDLYQEQVEVMLDVTDTSMDTYLEREWGNKPSDCVPYKDEELYDLPAPCTPLSLSCLQLSTPENRESPVVQAGGSKKHSNHLRKLVFDDFCDSSNVSNKDSSEDDISRSENEKKSECFSSPKTAFWDCCSTSYAQNLDFESSEGNTIANSVGEISSKLSEKSGSCVSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSNASPSKATKS

SEQ ID NO: 6 - protein, 726 aa, Macaca fascicularis
MAQTVQNVTLSLTLPITCHICLGKVRQPVICINNHVFCSICIDLWLKNNSQCPACRVPITPENPCKEIIGGTSESEPTLSHTVRKHLRKTRLELLHKEYEDEIDCLQKEVEELKSKNLSLESQIKTILDPLTLVQGNQNEDKHLVTDNPTKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICRRAPSADGKGSKGSEEDVASKNQGDSARKQPGSSTTSSSHLAKPSSSRLCDTSSARQESTSKAEINCSKNKDLYQKQVEIMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWECCSTSYAQNLDFESSEGTTIANSVGEIASKLSEKSGSCVSKRLNSIRSFEMNRTRTSSEASMDAAYLDKISELDSMMSESDNSKSPCNNGFKSLDLDGLSKSSQGSEFLEEPDKLEEKTKLNLSKGSLTNDQLENGNEWKPTSFFLLSPSDQEMNEDFSLHSSSCPVTDEIKPPSCLFQTEFSQGILLSSSHRLFEDQRFGSSLFKMSSEMHSLHNHLQSPWSTSFVPEKRNKNVNQSTKRKIQSSLSNASPSKATKS

SEQ ID NO: 7 - protein, 726 aa, Macaca mulatta; identical to SEQ ID NO: 6

SEQ ID NO: 8 - protein, 724 aa, Callithrix jacchus
MAQTVQNVTLSLTLPITCHICLGKVRQPVICINNHVFCSICIDLWLKNNSQCPACRVPITPENPCKEIIGGTSESEPMLSHTVRKHLRKTRFELLHKEYEDEIDCLQKEVEELKSKNLSLESQIKTILDPLTLVQGNQNENKHLVTDNPSKSTPETVAEWKKKLRTANEIYEKVKDDVDKLKEVNKKLKLENGGLVRENLRLKAEVDNRSPQKFGRFAVAALQSKVEQYERETNRLKKALERSDKYIEELESQVARLKNSSEEKEAMNSICQRALSADGKGSKGSEEDVASKNQGDSARKQPSSSTSSSHLPKPSSSRLCDSSARQENTSKAELNCSKNKDLYQKQVEIMLDVTDTSMDTYLEREWGNKPSDCVPYKGEELYDLPAPCTPLSLSCLHLSTPESRESSVVQVGGSKKHSNHLRKLVFDDFCDSSSVSNKDSSEDDISRSENEKKSECFTSPKTGFWDCCSTSFAQSLDFESSEGNTIANSVGEISSKLSEKSGSCISKRLNSIRSFEMNRTRTSSEASMDAAYLDKISELDSMMSESDNSKSPCNNGFKSLDLDGLSKSSQGSEFLEEPDKLEEKTKLNLSKGSLTNDQLENGNEWKPTSFFLLSPSDQEMNDDFSLHSGSSPGTNELKPPSCLFQTEFSQGILLSSSHRLFEDQRFGSSLFKMSSEMHSLHNHLQSPWSTSFVPEKRNKNVNQSTKRKIQSSLSNASPSKATKS

SEQ ID NO: 9 - protein, 717 aa, Equus caballus
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLMQGNQNEDKHPVADNPSKIDPETVAEWKKKLRTANEIYEKVKDDVDKLKEANKNLKLENGGLVRENLRLKAEVDNRSPQKFGRFTVAALQSKVEQYERETNRLKKALERSDKYIEELESQIAQLKNSSEEKEAMNSICQRTLSTDGKGSKCSEEDMASKNQGDGARKQLGSSTSSSHLAKPSSSSARQEGISKTEPNCSKNKDLYQKQVEIMLDVTDTSMDTYLEREWGNKPSDCVPYKDEELYDLPAPCTPLSLSCLQLSTPENRESSVVKAGGSKKHSNHLRKLVFDDFCDSSSVCNKDSSEDDRSENEKKSDCFTSPKTGFWDCCSTSYAQSLDFESSEGNTIANSVGEISSKLSEKSGSCLSKRLNSIRSFEMNRTRTSSEASMDAAYLDKISELDSMMSESDNSKSPCNNGFKSVDLDGLSKSSQGSEFLEESDKLEERTKPNLSKGSLTTDQLENGNEWKPTSFFLLSPSDQEMNEDFSLHPSSNPGTNEIKPPSCLFQTEFSQGVLLSSSHRLFEDQRFGSSLFKMSSEMHGLHNQLQSPWSTSFVPEKRNKNVNQSTKRKIQSSLSNASPSKATKS

SEQ ID NO: 10 - protein, 726 aa, Spermophilus tridecemlineatus
MAQTVQNVTLSLTLPITCHICLGKVRQPVICINNHVFCSICIDLWLKNNSQCPACRVPITPENPCKEIIGGTSESEPMLTHTVRKHLRKTRLELLHKEYEDEIDCLQKEVEELKSKNLSLESQIKTILDPLTLMQGSQNEDKHPVADNPSKIDSETVAEWKKKLRTANEIYEKVKDDVDKLKEANKKLKLENGGLVRENLRLKAEVDNRSPQKFGRFTVAALQSKVEQYERETNRLKKALERSDKYIEELESQVAQLKNSSEEKETIDSICQRALSADGNGSKSSEEDVASKNQGDSARKQPGSSASITVHLAKPSSGRISDTSSARQENTSKTESNCPKNKDMYQKQVEIMLDVTDTSMDTYLEGEWSNKPNDCVPFKDEELYDLPAPCTPLSLSCLQLNTPENRESSVVKAGSSKKHSNHLRKLVFDDFCDSQNVCNKDSSEDDTSRNENEKKSECFTSPKTGFWDCCSTSYAQSLDFENSEENTIANSVGEISLKLSEKSGSCLSKRLNCIRTFEMNRTRTSSEASMDAAYLDKISELDSMMSESDNSKSPCNNGFKSVDVDGLSKSSQGSEFLEESEKLEERTKLNLSKSSLTTDQLENGNEWKPTSFFLLSPSDQEMNEDFSLHSTSNPGTNDIKPPSCLFQTEFSQGVLLSSSHRLFEDQRFGSSLFKTSSEMHSLHNHLQSPWSTSFVPEKRNKNVNQSTKRKIQSSLSNASPSKATKS

SEQ ID NO: 11 - protein, 717 aa, Ailuropoda melanoleuca
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEDLKSKNLSLESQIKTILDPLTLMQGNQNEEKHPVADNPSKIDPETVAEWKKKLRTANEIYEKVKDDVDKLKEANKKLKLENGGLVRENLRLKAEVDNRSPQKFGRFTVAALQSKVEQHERETNRLKKALERSDKYIEELESQIAQLKNSSEEKEAMNSVCQRALSTDGKGSIGNEEDMASKNQGDGARKQLGSSTSSSHLAKPSSSSARQESTSKTEPNCSKNKDLYQKQVEIMLDVTDTSMDTYLEREWGNKRSDCIPYKDEELYDLPAPCTPLSLSCLQLSTPENRESPVVKAVGSKKHSNHLRKLVFDDFCDSPNVCNKDSSEDDRSENEKKSECFTSSKTGFWDCCSTSYAQNLDFESPEGNTIANSVGEITLKLSEKSGSCLSKRLNSIRSFEMNRTRTSSEASMDAAYLDKISELDSMMSESDNSKSPCNNGFKSVDLDGLSKSSQGSEFLEEPEKLEERTKANLSKGSLTTDQLENGNDWKPTSFFLLSPSDQEMNEDFSLHTSSNPGTNEIKPPSCLFQTEFSQGVLLSSSHRLFEDQRFGSSLFKMTSEMHGLHNHLQSPWSTSFVPEKRNKNMNQSTKRKIQSSLSNASPSKATKS

SEQ ID NO: 12 - protein, 717 aa, Canis familiaris
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEDLKSKNLSLESQIKTILDPLAVMQGNQNEDKHPVADNPSKIGPETEAEWKKKLRTANEIYEKVKDDVDKLKEANKKLKLENGGLVRENLRLKAEVDNRSPQKFGRFTVAALQSKVEQHERETNRLKKALERSDKYIEELESQIAQLKNSSEEKEAMNSICQRALSTDGKGSIGNEEDMASKSQGDGARKQVGSSTSSSHLAKPSSSSARQECTSKTEPNCSKNKDLYQKQVEIMLDVTDTSMDTYLEREWGNKPSDCIPYKDEELYDLPAPCTPLSLSCLQLSTPENRESSVVKAVGSKKHSNHLRKLVFDDFCDSPNVCNKDSSEDDRSENEKKSECFTSSKTGFWDCCSTSYAQNLDFESSEGNTIANSVGEISLKLSEKSGSCLSKRLNSIRSFEMNRTRTSSEASMDAAYLDKISELDSMMSESDNSKSPCNNGFKSVDLDGLSKSSQGSEFLEEPDKLEERTKPNLSKGSLTTDQLENGNEWKPTSFFLLSPSDQEMNEDFSLHTSSNPGTNEIKPPSCLFQTEFSQGVLLSSSHRLFEDQRFGSSLFKMTSEMHGLHNHLQSPWSASFVPEKRNKNMNQSAKRKIQSSLSNASPSKATKS

SEQ ID NO: 13 - protein, 725 aa, Oryctolagus cuniculus
MAQTVQNVTLSLTLPITCHICLGKVRQPVICINNHVFCSVCIDLWLKNNSQCPACRVPITPENPCKEIIGGTSESEPMLSHTVRKHLRKTRLELLHKEYEDEIDCLQKEVEELKSKNLSLESQIKTILDPLTLMQGNQNEDKHPVADNPNTVDPETIAEWKKKLRTANEIYEKVKDDVDKLKEANKKLKLENGGLVRENLRLKAEVDNRSPQKFGRFTVAALQSKVEQYERETNRLKKALERSDKYIEELESQVTQLKHSSEEKEAMDSICQRVLSADGKRIKDSEEDMVSKNQCDNARTQSGSSTSSSHLTKPSSSRLSDTSSARQESTSKTEPNSSKNKDLYQKRVEIMLDVTDANMDAYLEREWGNKPSDCVPYKDEELYDLPAPCTPLSLSCLQLNTPENRESSVVKAGSSRKHSNHLRKLVFDDFCDSTNVCNKDSSEDDISRSENEKKSECFTSPKTGFWDCCSTSYAQSLDFESSEGNNMANSVGEISSKLSEKSGSCLSKKLNSIRSFEINRTRTSSEASMDAAYLDKISELDSMMSESDNSKSPCNNGFKSVDLDGLSKSSQGSEFLEEPEKLEERTKLSLSKGSLTTDQLANGNEWKPASFFLLSPSDQEMNEDFSLHPTSNPGTNEIKPPSCLFQTEFSQNVLLSSSHRLFEDQRFGSSLFKMSSEMHGLGNHLQSPWSTSFVPEKRNKNMNQSTKRKIQSSLSNASPSKATKS

SEQ ID NO: 14 - protein, 727 aa, Otolemur garnettii
MAQTVQNVTLSLTLPITCHICLGKVRQPVICINNHVFCSICIDLWLKNNSQCPACRVPITPANPCKEIIGGTSESEPMLSHTVRKHLRKTRLELLHKEYEDEIDCLQKEVEELKSKNLSLESQIKTILDPLTLIQDNQNEDKHLVIDNSSKIDQETVAEWKKKLRTANEIYEKVKDDVDKLKEANKKLKLENGGLVRENLRLKAEVDNRSPQKFGRFTVAALQSKVEQYERETNRLKKALERSDKYIEELESQVAQLKKSGKEKEAMNSICQRSLSADGMGGKGSEEDVASKNQGDSASKQSGSSTSSSSHLAKASSSKLSDTGSARQESTINTDLNCSKNKDLYQKRVEIMLDVTDTHMDTYLESEWDNKPSDCVPYKDEELYDLPAPCTPLSLSCLQLSTPENRESSTVKAGGSIKHSNHLRKLVFDDFCESSSVSIKDSSEDDTNRNENDNKSECFTTPKTGFWDCCSTSYAQSLDFESSEGNTIANSVGEISSKLGEKPGSCLSKRLNSIRSFEMNRTRTSSEASMDAAYLDKISELDSMMSESDNSKSPCNNGFKSVDLDGLSKSSQGSEFLGESDNLEERTKLNLSKSSLTTADHLENGNEWKPTSFFLLSPSDQEMNEDFSLHPSSNPGTNETKPPSCLFQTEFSQGILLSSSHRLFEDQRFGSSLFKMSSEMHGLHNHLQSPWSTSFVPEKRNKNVSQSTKRKIQSSLSNASPSKATKS

SEQ ID NO: 15 - protein, 725 aa, Loxodonta africana
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLMQGNQNEDKHTVADNPSKIDSEAVAEWKKKLRTANEIYEKVKDDVDKLKEANKKLKLENGGLVRENLRLKAEVDNRSPQKFGRFTVAALQSKVEQYERETNRLKKALERSDKYIEELESQVAQLKNSSDEKEAVNSICQRVLSTDGSGNTGSEEDMASKSQGDGARKQPGSSTSSSSHLAKPSSSRLSDTSSVRQESTSKTESNHSNKDLYQKQVEIMLDVTDTSMDTYLESEWGNKASECVPYKDEELYDLPAPCTPLSLSCLQLSTPETRESSVVKAGGSKKHSNHLRKLVFDDFCDSPNVCNKDSSGDAISRSENEKKSDCFTSPKTRFWDCCSTSYAQSLGFESSEGNTIANSVGEMSSKSSEKSGSCLSKRLNSIRSFEMNRTRTSSEASMDAAYLDKISELDSMMSESDNSKSPCNNGFKSVDLDGLSKSAQSTEFLDEPDKLEERTKPNLSKGSLTTDQLENENEWKSTSFFLLPPSDQEMNEDFSLHPSSNPGTNEIKPPSCLFQTEFSQNVLLSSSHQLFEDQRFGSSLFKMSSEMHGLHNHLQSPWSTSFVAEKRNKNVNHSAKRKIQSSLSNASPSKATKS

SEQ ID NO: 16 - protein, 716 aa, Myotis lucifugus
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKNKNLSLESQIKTILDPLTLMQGNQNEEKHPVADNSSKIDPETVAEWKKKLRTANEIYEKVKDDVDKLKEANKKLKLENGGLMRENLRLKAEVDNRSPQKFGRFTVAALQSKVEQYERETNRLKKALERSDKYIEELESQVAQLKNSSEEKEAMNSICQRALSTDGKGCKGSEEDMASTNQGDGARKQLGSSTPSSHLTQPSNSSARQESTSKTELNCSKNKDLYQKQVEIMLGVTDASMDTYLEREWGNKPTDSTPYKDELYDLPAPCTPLSLSCLQLSTPENRESSMAKAGSSKKHSNHLRKLVFDDFCNSTNVCNKDSSEDDKSENEKKSECFTSPKTGFWDCCSTSYAQSLDFESAEGNTIANSVGEISSKLSEKSGSCLSKRLNSIRSFEMNRTRTSSEASMDAAYLDKISELDSMMSESDNSKSPCNNGFKSVDLDGLSKSSQGSEFLEEPDKLEERTKPNLSKGSLTSDQLENGNEWKPSSFFLLSPSDQEMSEEFSLHPSSNSGTNELKPPSCLFQTEFSEGVLLSSSHRLFEDQRFGSSLFKMSSEMQGLPNHLQSPWSTSFVPEKKNKNVNQSTKRKIQSSLSNASPSKATKS

SEQ ID NO: 17 - protein, 724 aa, Heterocephalus glaber
MAQTVQNVTLSLTLPITCHICLGKVRQPVICINNHVFCSICIDLWLKNNSQCPACRVPITPENPCKEIIGGTSESEPMLSHAVRKHLRKTRLELLHKEYEDEIDCLQKEVEELKSKNLSLESQIKAILDPLTLMQGNQNEDKHPVADNSSKIDLETVEEWKKKLRTANEIYEKVKDDVDKLKEANKKLKLENGGLVRENLRLKAEVDNRSPQKFGRFTVAALQSKVEQYERETSRLKKALERSDKYIEELESQVAQLKTSGEEKEAMDAICQRALSADGKGGSKEDVESNSQGDSARKQPRLSTSSSSNLAKPCISRLSDTSTARQESTSKTEPNHPKNKDLYQKPVEIMLDVTDTSMDTYLEGEWSNKPSNCVPYKDEELYDLPAPCTPLSLSCLQLNTPENRESSVVKAGGSKKHSNHLRKLVFDDFCDSSNVCTKECSEADISRNENEKKSECFASPKTGFWDCCSTSYAQSLDFENSEGNIIANSVGEICPKLIEKASSCLSKRLNSIRSFEMNRTRTSSEASMDAAYLDKISELDSMMSESDNSKSPCNNGFKSVDLDGLSKSSQGNEFLEEPVKLEERTELKLRKGSLTADQLENGNEWKPTSFFLLSPSDQEMNEDFALHPTSNPETNEIKPPSCLFQPEFSQGVLLSSSHGLFEDQRFGSSLFKLSSEMHSLHNHLQSPWSASFVPEKRNKNVNQSTKRKIHSSLSNASPSKATKS

SEQ ID NO: 18 - protein, 751 aa, Pteropus alecto
MALLGCRSRPRAVPVAARIRGRRDFRVGGILNAAMAQTVQNVTLSLTLPITCHICLGKVRQPVICINNHVFCSVCIDLWLKNNSQCPACRVPITPENPCKEIIGGTSESEPMLSHTVRKHLRKTRLELLHKEYEDEIDCLQKEMEELKSKNLSLESQIKTILDPLIFTQGNQSEDKHPVVDNPSKIDPETVAEWKKKLRTANEIYEKVKDDVDKLKEANKKLKLENGGLMRENLRLKAEVDNRSPQKFGRFTIAALQSKVEQYERETNRLKKALERSDKYIEELESQIAQLKNSSEEKEAMNSICQNTLSTDDKGSKGSKEDMPSKNQGDGARKQLCSATSSSHLAQPSNSSARQESTSKTEPNCSKNKDLYQKQVEIMLDVTDTSMDTYLEREWGNKPSDCAPYKAEELYDLPAPCTPLSLSCLQLSTPENRENSVGKAGCSKNHSNHLRKLVFDDFCDSPNVCNKESSEDGRSENEKKSECFPSPKTGFWDCCSTSYAQSLDFENSEGNLIANSVGEISSKFSEKSGSCLSKRLNSIRSFEMNRTRTSSEASMDAAYLDKISELDSMMSESDNSKSPCNNGFKSVDLDGLSKSSQGSEFLEEPDELEERTKPDLSKGSLTTGQLENGNEWKPTSYFFLSPSDQEMNEDFSLHPSSDSGTTEIKPPSCLFQTEFSQSVLLSSSHRLFEDQRFESSLFKMSSEMHGLHNHLQSPWSTSFVPEKRNKNVNQSTKRKIQSSLSNASPSKATKS

SEQ ID NO: 19 - protein, 717 aa, Bos grunniens
MAQTVQNVTLSLTLPITCHICLGKVRQPVICINNHVFCSVCIDLWLKNNSQCPACRVPITPENPCKEIIGGTSESEPMLSHTVRKHLRKTRLELLHKEYEDEIDCLQKEVDELKSKNLSLESQIKTILDPLTLMQGNQNEDKLPVADNPSKIDPETVAEWKKRLRTANEIYEKVKDDVDKLKEANKKLKLENGGLVRENLRLKAEVDNRSPQKFGRFTVAALQSKVEQYERETNRLKKALERSDKYIEELESQITQLKNSSDEKEAMNAICQRALSTDGKGSKGTEEDMASKNQGDGARKQLGSATSSSHLAKPSSSSVRQEGTSKTEPNCSKNQDLYQKRVETMLTVTDTSMDTYLEREWGNKPSDCMPYKDEELYDLPASCAPLSLSCLQLNTPDNRESSVVKAGGSKKQSNHLRKLMFDEFCDSPNVCNKDSSEDDRSESEKKSECFTSPKTGFWDCCSTSYAPSLDFENSEENTIANCVGEISSKLSEKPGSCLSKRLNSLRSFEMNRTRTSSEASMDAAYLDKISELDSMMSESDNSKSPCNNGFKSLDLDGLSKSSQCSEFLEETDKLEERTKPNLSKGSLTTDQLENGNEWKPNSFFLLSPSDQEMNEDFSLHSSSNPGTNEIKPPSCLFQTEFSQGVLLSSTNRLFEDQRFGSSLFKMSSEMPGLHTHLQSPWSTSFVPEKRNKNVNQSAKRKIQSSLSNASPSKATKS

SEQ ID NO: 20 - protein, 717 aa, Bos taurus; identical to SEQ ID NO: 19

SEQ ID NO: 21 - protein, 723 aa, Cavia porcellus
MAQSVQNVTLSLTLPITCHICLGKVRQPVICINNHVFCSICIDLWLKNNSQCPACRVPITPENPCKEIIGGASESEPMLSHTVRKHLRKTRLELLHKEYEDEIDCLQKEVEELKNKNLSLESQIKTILDPLTLMQGNQNEDKLPVADNPSKIDPQTVSEWKKKLRTANEIYEKVKDDVDKLKEVNKKLKLENGGLVRENLRLKAEVDNRSPQKFGRFTVAALQSKVEQYERETNRLKKALERSDKYIEELESQIAQLKNSSEERETMDAICQRALSADGKGGSEEDVASNNQGDSARKPSSSTLSSSHLGKLSSSRLSDTSAARQEITCKTEPNQHKNKDLYQKQVEIMLDVSDTSMNTYLEGEWGGKPSNCVAYKDEELYDLPAPCTPLSLSCLQLNTPENRESSAVKAGGSKKHSNHLRKLVFDDFCESPNVCNKDCSEDDVSRNENEKKSECFAPPKTAFWDCCSTSYAQSLDFENSEGNIIANSVGEIPSKLSEKSTSCLSKRLNSIRSFEMNRTRTSSEASMDAAYLDKISELDSMMSESDNSKSPCNNGFKSVDLDGLSKSSQGNDFLEEPSKLDERTELNLPKSSLTADQLKNGNEWKPTSFFLLSPSDQEMNEDFSLHSTSNPETNEIKPSSCLFQTEFSQGVLLSSSHRLFEDERFGSSLFKMSSEMHSLHNHLQSPWSASFVPEKRNKNVNQSTKRKIHSTLSNASPSKATKS

SEQ ID NO: 22 - protein, 717 aa, Sus scrofa
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEIEELKSKNLSLESQIKTILDPLTLMQGNQNEDKHPVADNLSKVDPETVAEWKKKLRTANEIYEKVKDDVDKLKEANKKLKLENGGLVRENLRLKAEVDNRSPQKFGRFTVAALQSKVEQYERETNRLKKALERSDKYIEELESQITQLKNSSDEKEARNSICQRALSTDGKGNTGSEEDMASKNQGDGARKQLGSSTSSSHLAKPSSSSARQEGTNKTEPSCCKNKDLYQKRAEIMLDVTDTSMDTYLEREWGGKPSDCVAYKDEELYDLPAPCTPLSLSCLQLSTPENRESSVVKAGVSKKHPNHLRKLVFDDFCDSPGVSNKDSSEDDRSESEKKSECFTSPKTGFWDCCSPSYTPSLDFESSEGTIANSVGEFLKLSEKSGSCLSKRLNSIRTFEMNRTRTSSEASMDAAYLDKISELDSMMSESDNSKSPCNNGFKSLDLDGLSKSSQGSEFLEEPDKLEERTKPHLSKGSLTTDQLENGNEWKSNSFFLLSPSDQEMNEDFSLHPSSNPGTNEIKPPSCLFQTEFSQGVLLSSSHRLFEDQRFGSSLFKMSSEMHGLQNHLQSPWSTSTSFVPEKRNKNMNQSTKRKIQSSLSNASPSKATKS

SEQ ID NO: 23 - protein, 722 aa, Mus musculus
MAQTVQNVTLSLTLPITCHICLGKVRQPVVCTNNHVFCSICIDLWLKNNSQCPACRVPITPENPCKEIIGGTSESEPMLSHTVRKHLRKTRLELLHREYEDEIDCLQKEVEELKSKNLSLESQIKTILDPLALMQGSQNEDKHPLADNPSKMDPDSVVEWKKKLRTANEIYEKVKDDVDKLKEANKKLKLENGGLLRENLRLKAEVDNRSPQKFGRFTVAALQSKVEQYERETNRLKKALERSDKYIEELESQVAHLKHSEEAKEDVDALCQRAPSADSKGPNGSDELGPPKNQSDSARKQAGSASASHLASPSSSRLADSGSVRQESTSRTEPNCPQNKDRYPKPTEPRLGARETPMDTYLEREWGSKPSDCAPYKEDELYGIPASCTPLSLSCLQLNTPENRENPVIKAGSSKKHANHLRKLVFDDFCDSPNACNNNSSEDDRRENEKKSDCFASSKTGFWDCCSTSYAQSLEFDGSEGNAIANSVGEIPSKLSEKSGSCLSKRLSCIRSLEMNRTRTSSEASMDAAYLDKISELDSMMSESDNSKSPCNNGFKSVEVEGPSKSPQGREFLEEPDKLQEGSKLNLSKPALTADGLESGGEWKPSSFFLLSPADHEMSEDFSLHSTSHSGTSEVKPPNCLFQTEFSQGALLSSSQGLFEDQRFGSSLFKMSSEMQSLHSPLQSPWSAAFVPEKRSKNGNQSTKRKIQSSLANASPSKATKS

SEQ ID NO: 24 - protein, 659 aa, Xenopus laevis
MAQTVQNVTLSLTLPITCHICLGKVRQPVICVNHHVFCTVCIELWLKNNNQCPVCRVPITAENPCKDIIGGTGENECALDHSVRKHLRKTRLELLHNEYEEEIESLLKEREELKQKNVTLEKERSPTTTVSLGHDCNCGTKQGTENTSSEQSTSEKWKKKLEETNAASRKLVADVEKLKEENTKLINENIDYVRENFRLKTEVDSRSPQKFGRFTVAALHAKIDQYEKEMNRLKKALERSDKYIEELEVQVDQLKRPADWKPNERSHNENPVRDKETVNSEIGDSSLNYEGVKLEQMDKIPEKAEKNQEDSNSTCPTANNHKFIVNKSEYLSRTGCSMDFQKDRKSFPSSSSATWKESLKSPHGKNSSRNKPRESNTPCKEKEIHEFSSPGTSLSFSSLQLNSPDCKFTLPSNQVNAKKPLTHLRKLVFDDSPKKRKPNGFSNANNNGGLSKPFNEPKSEYWACEVKHKHSRDKLKESYVQLESLKNKEGQILQALPSLSSEEGNRTTTTSESSMDITFLDVNKLDSMMSTLEGNENPHYHSVKSDAVMHSNSQPRNGLQALYSNPHSQCAGGLLSHESRSSDKSHNNDFHTSRSTSSLLTPFYATAQSYSSKNLMDLMAPEMTSNSGSTSYTDNKNGLTSKRKLFNPICDSPPKSFKP

SEQ ID NO: 25 - protein, 717 aa, Felis catus
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEDLKSKNLSLESQIKTILDPLTLMQSSQTEDKHPIADNPSKIDPETVAEWKKKLRTANEIYEKVKDDVDKLKEANKKLKLENGGLVRENLRLKAEVDNRSPQKFGRFTVAALQSKVEQHERETNRLKKALERSDKYIEELESQIAQLKNSSEEKEAINSICQRALSTDGKGSLGGEEDMASKNQGDGARKQLGSSTSSSHLPKPSSSSARQESTSKTEPNCSKNKDLYQKRVEIMLDVTDTSMDTYLEREWGNKPSDCIPYKDEELYDLPAPCTPLSLSCLQLSTPENRESSVVKAVGSKKHSNHLRKLVFDDFCDSSNVCTKDSSEDDRSENEKKSECFTSSKTGFWDCCSTSYAQSLDFESSEGNTIANSVGEISLKLSEKSGSCISKRLNSIRCFEMNRTRTSSEASMDAAYLDKISELDSMMSESDNSKSPCNNGFKSVDLDGLSKSSQGSEFLEEPDKLDERTKPNLSKGSLTTDQLENGNEWKPTSFFLLSPSDQEMNEDFSLHTSSNPGTNEIKPPSCLFQTEFSQGVLLSSSHRLFEDQRFGSSLFKMTSEMHGLHNHLQSPWSTSFVPEKRNKNITQSTKRKIQSSLSNASPSKATKS

SEQ ID NO: 26 - protein, 724 aa, Rattus norvegicus
MAQTVQNVTLSLTLPITCHICLGKVRQPVVCTNNHVFCSICIDLWLKNNSQCPACRVPITTENPCKEIIGGTSESEPMLSHTVRRHLRRTRLELLHREYEDEIDCLQKEVEELKSKNLSLESQIKTILDPLALMQGSQNEDKHPMADNPSKIDPESVVEWKKKLRTANEIYEKVKDDVDKLKEANKKLKLENGGLLRENLRLKAEVDNRSPQKFGRFTVAALQSKVEQYERETNRLKKALERSDKYIEELESQVAQLRHSEETEEGMGALCQRAPSADGKGPKGSDDVAASKNPSEGAGKQAGSANSASHLATPSSSRLADTSSARQESTSRTEPNCLQNKDRYPKPTEAQLGARETPMDTYLERQWGSKPSDCAPYKEEELYGLQASCTPLSLSCLQLNTPENRENPVIKAGGSKKHANHLRKLVFDDFCDSPNTCNNNSSEDDRSGNEKKSDCFASSKTEFWDCCSTSYAQSLEFDGSEGNTIANPVGEVPSKLSEKSGFCLSKRLSCIRSLEMNRTRTSSEASMDAAYLDKISELDSMMSESDNSKSPCNNGFKSVESEGSSKSPQGREFLEEPDKLQEGAKLSLPKNALEADGLEGGSEWKPSSFFLLSPSDHEMSEDFSLHTTSHSGASEVKSQNCLFQTEFSQGVQLLSSSQGLFEDQRFGSSLFKMSSDMQSLPGPLQSPWSAAFVSEKRSKNGNQSAKRKIQSSLSNASPSKATKS

SEQ ID NO: 27 - protein, 654 aa, Xenopus tropicalis
MAQNVQNVTLSLTLPITCHICLGKVRQPVICVNYHVFCTVCIELWLKNNSQCPVCRVPITAENPCKDIIGGTGENECSLDHSVRKHLRKTRLELLHNEYEEEIESLLKELEELKQKNIILERERSPTTTVSLGIDCNCGTKLRTENTSSEQTTSEKWKKKLEETNAASRKLSADVEKLKEENAKLINENIDYVRENFRLKTEVDSRSPQKFGRFTVAALHAKIDQYEKEMNRLKKALERSDKYIEELEVQVDQLKRAADLKQDESSQNENSVHDKDTVNSEIGCTELYEGVKLEQMGEKPEKHDINKENSSDTCPAANNEKSHVHRSEYLTQTGCSLNFQKERKMFLSSGSAMWKESMKGPHDNITSQNKQTETYTPTKEKEIHEYNSPSTSLSFSSLQLNSPDCKLSLPSNQVNAKKPLTYLRKLVFEDYPKKGKPNSFSSANNNIPKPFNEQKSEYMTCEVKHKHQRGNLKESDMQSEGQILQAFSLPSSGEVHHTTTTSESPMDTFLHNVNNLDSLMSTLEGNKSLHYHSVTADAVMHLNSQPGNGLQAFFSNPHTRDGHFPLESQSSDKSHINDFHTSGRTSSLVLPFNDMPQIKYQNTTESSNILTNLNASELASNSCCTSYTNNKHGPASKRKLLNTICESPPKTMKP

SEQ ID NO: 28 - protein, 39 aa, Artificial Sequence
  ring finger - zinc finger of OBI1 proteins
CHICLGKVRQPVXCXNNHVFCSXCIDLWLKNNSQCPACR

SEQ ID NO: 29 - protein, 194 aa, Artificial Sequence
  Coiled-Coil domain of huaman Obi1
VRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALS

SEQ ID NO: 30 - protein, 450 aa, Artificial Sequence
  C-terminal part of human Obi1
ADGKGSKGSEEDVVSKNQGDSARKQPGSSTSSSSHLAKPSSSRLCDTSSARQESTSKADLNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWDCCSTSYAQNLDFESSEGNTIANSVGEISSKLSEKSGLCLSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSSASPSKATKS

SEQ ID NO: 31 - DNA, 3552 bp, Homo sapiens
ggcgaagagtagcggtaggtcggcgggacttccgtgttggcgggattctgaacgctgccatggctcagaccgtgcagaatgttacattgtcgctcactctgcccatcacgtgccacatttgcttggggaaggtacgtcagcctgtcatatgcatcaacaaccatgtattttgttcgatttgtattgatttgtggttgaagaataatagccagtgtccagcttgcagagtccccatcactcctgaaaatccttgcaaagaaattataggaggaacaagtgaaagtgaacctatgctaagccatacggtcaggaagcatcttcggaaaactagacttgaattactacacaaagaatatgaggacgaaatagattgtttacagaaagaagtagaagagcttaagagtaaaaatctcagcttggagtcacagatcaaaactattctggatcctttaaccttggtgcagggcaaccaaaatgaagacaaacatctagtcacagataatccaagtaaaattaacccagaaactgtagcagagtggaagaaaaaactcagaacagctaatgaaatctatgaaaaagtgaaagatgatgtggataagctaaaggaggcaaataaaaaattgaaattggaaaatggtggtctggtgagggagaatttacgactgaaggctgaagttgataacagatcacctcaaaagtttggaaggtttgcagttgctgctcttcagtccaaagtagaacagtatgagcgtgaaaccaatcgcctcaagaaagccctggaacgaagtgataagtatatagaggaactagaatctcaagttgcacagctaaaaaattcaagtgaagagaaagaagctatgaattccatttgccagacagcactttctgcagatggcaaagggagcaaaggcagtgaggaggatgtggtgtcaaagaatcaaggcgatagtgccagaaagcagcctggctcatccacctccagttcttctcacctagcgaagccttccagcagcagactgtgtgacaccagttctgcaaggcaggaaagtaccagcaaagcagaccttaactgttctaagaacaaagacctatatcaagaacaggtagaagtaatgttagatgtgacagatacaagtatggatacttatttggaaagagaatgggggaataaaccaagtgactgtgtaccctacaaagatgaagaactttatgatcttccagctccttgtactcctttgtcccttagttgccttcagctcagtactccagaaaatagagagagctctgtggtccaagcaggaggttccaaaaagcactcaaaccatctcagaaaattggtgtttgatgatttttgtgattcttcaaatgtttctaataaagattcttcagaagatgatataagtagaagtgaaaatgaaaagaaatcagaatgtttttcttccccaaagacaggattttgggactgttgttccacaagctatgcccaaaacttagattttgaaagttcagaggggaacacgatagcaaattctgttggagaaatatcttcaaaattgagtgagaaatcaggcttatgtttatccaaaaggttgaattctattcgctcttttgaaatgaaccggacaagaacatccagtgaagcatcgatggatgctgcttaccttgacaaaatctctgagttggattcaatgatgtcagagtcagacaacagcaagagcccttgtaataacggttttaagtcactggatttggatgggttatcaaagtcatctcaaggcagtgaatttcttgaggaacctgataagttggaagaaaaaactgagctaaacctttccaaaggttctctaactaatgatcagttagaaaatggaagtgaatggaaacccacttctttttttctcctctctccatctgaccaagaaatgaatgaagatttttcactccattccagttcttgtccagtaactaatgaaatcaaacccccaagctgcttgtttcagacagagttttcccagggcattttgttaagcagttcacatcgactatttgaagatcaaagatttgggtcatctttgtttaagatgtcctcagagatgcacagtcttcataaccaccttcagtctccttggtctacttcctttgtgcctgaaaagaggaataaaaatgtgaatcaatcaacaaaaagaaaaatccagagcagcctttccagtgccagcccatcaaaagcaactaaaagttgactcattagaaaggtgtcatttgtggttttgtcctgagagaaatagaaaagttgttaaagttaccttttttcctcataaaagttctatacaaattggaattgataatctttagtcaagtatcaagtcaggatggtggattaacctgtacccagaatacttattgttcattttgaaaagactttgttcttttcatttttatttgggagtctttgtgaccagagaagttagggaggaggttatttttgtgttttggggttggttggttggttggttttgtttttggttttgtttttttactgaatttgatatgtatctcggttggatatacattgtttttttaaaaaatgttatttaactgttagatacagtggcctgttgataagccccacttgtcttcagaacttggatttcttaaataaaacttttagtgttgtctatacactgctcaataagacacttgagtttaagcttttcccagggtggaaattattttacctgtccctttttatttatgtttagtgatggcctagtttttctgcagggccatgatggagaaatagcactctagccttagtccaatattgatttactttctttttttaggttttatgtatatgtttgcattttttagcattgtgttttgtccagttttgtgaaaatgttctgctagtatgaaagaaaacattttctatatgaagacatttgttttatgttaggtagcttacattttctcctctgcgtgtgtgtgtatgtgtgtaaaatcagaaatttagcatactatggaaagaaggcatggagcacttgggtttagaggaacctaaaacatcatagcttcattgttccagatgtaacaggtttgaaagagctcatcgccaagttcttgatccacttgcattccaggggagttttcttttgagtagtatgtttcttgtttgcatgttcctgttctttgtggaaactatgcatggtagcatttttgcttgctgtgttttccatacttaagaaaaagaggtttcagttggctgatagaatatcttttatgtaggacaaaacttttctgtgaagagtgttgagggggtgaagataggtaagaggtaagcacaatttttaatttaggctctgaaaaagtgtattgttctaaacgtatttggtatgcctatataggtctttaaaaatgggtatgtatgctgtttaatgtgcactgaacattttacattaatattgtactgttttacattaatactgcatgcttttctatgtgaattgaataaagaatgtcataagcactgtgttcaaaaaaaaaaaaaaaaaa

SEQ ID NO: 32 - DNA, 19 bp, Artificial sequence
  siRNA inhibiting OBI1
gtagaagtaatgttagatg

SEQ ID NO: 33 - DNA, 19 bp, Artificial sequence
  siRNA inhibiting OBI1
ccaaaggttctctaactaa

SEQ ID NO: 34 - DNA, 19 bp, Artificial sequence
  siRNA inhibiting OBI1
ggatttggatgggttatca

SEQ ID NO: 35 - DNA, 19 bp, Artificial sequence
  siRNA inhibiting OBI1
gaacgaagtgataagtata

SEQ ID NO: 36 - RNA, 19 nt, Artificial sequence
  siRNA inhibiting OBI1
guagaaguaauguuagaug

SEQ ID NO: 37 - RNA, 19 nt, Artificial sequence
  siRNA inhibiting OBI1
ccaaagguucucuaacuaa

SEQ ID NO: 38 - RNA, 19 nt, Artificial sequence
  siRNA inhibiting OBI1
ggauuuggauggguuauca

SEQ ID NO: 39 - RNA, 19 nt, Artificial sequence
  siRNA inhibiting OBI1
gaacgaagugauaaguaua

SEQ ID NO: 40 - DNA, 19 bp, Artificial sequence
  siRNA inhibiting OBI1
catctaacattacttctac

SEQ ID NO: 41 - DNA, 19 bp, Artificial sequence
  siRNA inhibiting OBI1
ttagttagagaacctttgg

SEQ ID NO: 42 - DNA, 19 bp, Artificial sequence
  siRNA inhibiting OBI1
tgataacccatccaaatcc

SEQ ID NO: 43 - DNA, 19 bp, Artificial sequence
  siRNA inhibiting OBI1
tatacttatcacttcgttc

SEQ ID NO: 44 - RNA, 19 nt, Artificial sequence
  siRNA inhibiting OBI1
caucuaacauuacuucuac

SEQ ID NO: 45 - RNA, 19 nt, Artificial sequence
  siRNA inhibiting OBI1
uuaguuagagaaccuuugg

SEQ ID NO: 46 - RNA, 19 nt, Artificial sequence
  siRNA inhibiting OBI1
ugauaacccauccaaaucc

SEQ ID NO: 47 - RNA, 19 nt, Artificial sequence
  siRNA inhibiting OBI1
uauacuuaucacuucguuc

SEQ ID NO: 48 - DNA, 21 bp, Artificial sequence
  siRNA inhibiting OBI1
guagaaguaauguuagaugtt

SEQ ID NO: 49 - DNA, 21 bp, Artificial sequence
  siRNA inhibiting OBI1
ccaaagguucucuaacuaatt

SEQ ID NO: 50 - DNA, 21 bp, Artificial sequence
  siRNA inhibiting OBI1
ggauuuggauggguuaucatt

SEQ ID NO: 51 - DNA, 21 bp, Artificial sequence
  siRNA inhibiting OBI1
gaacgaagugauaaguauatt

SEQ ID NO: 52 - DNA, 21 bp, Artificial sequence
  siRNA inhibiting OBI1
caucuaacauuacuucuactt

SEQ ID NO: 53 - DNA, 21 bp, Artificial sequence
  siRNA inhibiting OBI1
uuaguuagagaaccuuuggtt

SEQ ID NO: 54 - DNA, 21 bp, Artificial sequence
  siRNA inhibiting OBI1
ugauaacccauccaaaucctt

SEQ ID NO: 55 - DNA, 21 bp, Artificial Sequence
  siRNA inhibiting OBI1
uauacuuaucacuucguuctt

SEQ ID NO: 56 - RNA, 10 nt, Artificial Sequence
  shRNA loop
cuuccuguca

SEQ ID NO: 57 - protein, 861 aa, Homo sapiens
MAHYPTRLKTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAKCQKPVRAKSKSAESPSWTPAEHVAKRIESRHSASKSRQTPTHPLTPRARKRLELGNLGNPQMSQQTSCASLDSPGRIKRKVAFSEITSPSKRSQPDKLQTLSPALKAPEKTRETGLSYTEDDKKASPEHRIILRTRIAASKTIDIREERTLTPISGGQRSSVVPSVILKPENIKKRDAKEAKAQNEATSTPHRIRRKSSVLTMNRIRQQLRFLGNSKSDQEEKEILPAAEISDSSSDEEEASTPPLPRRAPRTVSRNLRSSLKSSLHTLTKVPKKSLKPRTPRCAAPQIRSRSLAAQEPASVLEEARLRLHVSAVPESLPCREQEFQDIYNFVESKLLDHTGGCMYISGVPGTGKTATVHEVIRCLQQAAQANDVPPFQYIEVNGMKLTEPHQVYVQILQKLTGQKATANHAAELLAKQFCTRGSPQETTVLLVDELDLLWTHKQDIMYNLFDWPTHKEARLVVLAIANTMDLPERIMMNRVSSRLGLTRMCFQPYTYSQLQQILRSRLKHLKAFEDDAIQLVARKVAALSGDARRCLDICRRATEICEFSQQKPDSPGLVTIAHSMEAVDEMFSSSYITAIKNSSVLEQSFLRAILAEFRRSGLEEATFQQIYSQHVALCRMEGLPYPTMSETMAVCSHLGSCRLLLVEPSRNDLLLRVRLNVSQDDVLYALKDE

SEQ ID NO: 58 - protein, 647 aa, Homo sapiens
MGPLSARLLMQRGRPKSDRLGKIRSLDLSGLELLSEHLDPKLLCRLTQLQELDLSNNHLETLPDNLGLSHLRVLRCANNQLGDVTALCQFPKLEELSLEGNPFLTVNDNLKVSFLLPTLRKVNGKDASSTYSQVENLNRELTSRVTAHWEKFMATLGPEEEAEKAQADFVKSAVRDVRYGPESLSEFTQWRVRMISEELVAASRTQVQKANSPEKPPEAGAAHKPRARLAALKRPDDVPLSLSPSKRACASPSAQVEGSPVAGSDGSQPAVKLEPLHFLQCHSKNNSPQDLETQLWACAFEPAWEEGATSQTVATCGGEAVCVIDCQTGIVLHKYKAPGEEFFSVAWTALMVVTQAGHKKRWSVLAAAGLRGLVRLLHVRAGFCCGVIRAHKKAIATLCFSPAHETHLFTASYDKRIILWDIGVPNQDYEFQASQLLTLDTTSIPLRLCPVASCPDARLLAGCEGGCCCWDVRLDQPQKRRVCEVEFVFSEGSEASGRRVDGLAFVNEDIVASKGSGLGTICLWSWRQTWGGRGSQSTVAVVVLARLQWSSTELAYFSLSACPDKGIVLCGDEEGNVWLYDVSNILKQPPLLPAALQAPTQILKWPQPWALGQVVTKTMVNTVVANASFTYLTALTDSNIVAIWGRM

SEQ ID NO: 59 - protein, 43 aa, Artificial Sequence
  Ring finger domain of humain Obi1
TCHICLGKVRQPVICINNHVFCSICIDLWLKNNSQCPACRVPI

SEQ ID NO: 60 - protein, 259 aa, Artificial Sequence
  RING + Coil-coiled domain
TCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTAL

SEQ ID NO: 61 - protein, 237 aa, Artificial Sequence
  RING domain and coil-coiled domain
TCHICLGKVRQPVICINNHVFCSICIDLWLKNNSQCPACRVPIVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALS

SEQ ID NO: 62 - protein, 493 aa, Artificial Sequence
  Ring and C-ter domain
TCHICLGKVRQPVICINNHVFCSICIDLWLKNNSQCPACRVPIADGKGSKGSEEDVVSKNQGDSARKQPGSSTSSSSHLAKPSSSRLCDTSSARQESTSKADLNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWDCCSTSYAQNLDFESSEGNTIANSVGEISSKLSEKSGLCLSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSSASPSKATKS

SEQ ID NO: 63 - protein, 644 aa, Artificial Sequence
  Coil-coiled domain and Cter domain
VRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALSADGKGSKGSEEDVVSKNQGDSARKQPGSSTSSSSHLAKPSSSRLCDTSSARQESTSKADLNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSECFSSPKTGFWDCCSTSYAQNLDFESSEGNTIANSVGEISSKLSEKSGLCLSKRLNSIRSFEMNRTRTSSEASMDAAYLDKISELDSMMSESDNSKSPCNNGFKSLDLDGLSKSSQGSEFLEEPDKLEEKTELNLSKGSLTNDQLENGSEWKPTSFFLLSPSDQEMNEDFSLHSSSCPVTNEIKPPSCLFQTEFSQGILLSSSHRLFEDQRFGSSLFKMSSEMHSLHNHLQSPWSTSFVPEKRNKNVNQSTKRKIQSSLSSASPSKATKS

SEQ ID NO: 64 - DNA, 35 bp, Artificial sequence
  derived from ORC1
ctggcggccgcaccatggcacactaccccacaagg

SEQ ID NO: 65 - DNA, 30 bp, Artificial sequence
  derived from ORC1
ctggcggccgcctcgtctttcagcgcatac

SEQ ID NO: 66 - DNA, 40 bp, Artificial sequence
  derived from  ORC2
ctggcggccgcaccatgagtaaaccagaattaaaggaaga

SEQ ID NO: 67 - DNA, 32 bp, Artificial sequence
  derived from ORC2
ctggcggccgcagcctcctcttcttccttttc

SEQ ID NO: 68 - DNA, 30 bp, Artificial sequence
  derived from LRWD1
ctggcggccgcaccatgggccccctctcgg

SEQ ID NO: 69 - DNA, 28 bp, Artificial sequence
  derived from LRWD1
ctggcggccgccatcctcccccagatgg

SEQ ID NO: 70 - DNA, 50 bp, Artificial sequence
  derived from FLAG tag
gatccgccaacatggactacaaggacgacgatgacaagtccatggctggg

SEQ ID NO: 71 - DNA, 50 bp, Artificial sequence
  derived from FLAG tag
aattcccagccatggacttgtcatcgtcgtccttgtagtccatgttggcg

SEQ ID NO: 72 - DNA, 34 bp, Artificial sequence
  derived from ORC1
ctgtctagagccatggcacactaccccacaaggc

SEQ ID NO: 73 - DNA, 31 bp, Artificial sequence
  derived from ORC1
ctgtctagattactcgtctttcagcgcatac

SEQ ID NO: 74 - DNA, 35 bp, Artificial sequence
  derived from ORC2
ctgccatgggaagtaaaccagaattaaaggaagac

SEQ ID NO: 75 - DNA, 30 bp, Artificial sequence
  derived from ORC2
ctgctcgagtcaagcctcctcttcttcctt

SEQ ID NO: 76 - DNA, 22 bp, Artificial sequence
  derived from ORC3
ctgccatggctacgtcctcgat

SEQ ID NO: 77 - DNA, 30 bp, Artificial sequence
  derived from ORC3
ctgtctagattagcagcctccccatgttag

SEQ ID NO: 78 - DNA, 29 bp, Artificial sequence
  derived from ORC4
ctgctcgagagcagtcgtaaatcaaagag

SEQ ID NO: 79 - DNA, 32 bp, Artificial sequence
  derived from ORC4
ctgtctagattataaccagcttagtgaggatg

SEQ ID NO: 80 - DNA, 29 bp, Artificial sequence
  derived from ORC5
ctggaattctccccacttggaaaacgtgg

SEQ ID NO: 81 - DNA, 33 bp, Artificial sequence
  derived from ORC5
ctgctcgagtcacaagaaatcatacaagtattt

SEQ ID NO: 82 - DNA, 25 bp, Artificial sequence
  derived from ORC6
ctgctcgaggggtcggagctgatcg

SEQ ID NO: 83 - DNA, 31 bp, Artificial sequence
  derived from ORC6
ctgtctagattactctgctgtagccttttga

SEQ ID NO: 84 - DNA, 18 bp, Artificial sequence
  derived from obi1
ctgccatggctcagaccg

SEQ ID NO: 85 - DNA, 34 bp, Artificial sequence
  derived from obi1
ctgtctagattaacttttagttgcttttgatggg

SEQ ID NO: 86 - DNA, 27 bp, Artificial sequence
  derived from obi1
ctgccatggcttgcagagtccccatca

SEQ ID NO: 87 - DNA, 32 bp, Artificial sequence
  derived from obi1
ctgtctagattatttgccatctgcagaaagtg

SEQ ID NO: 88 - DNA, 26 bp, Artificial sequence
  derived from obi1
ctgccatggggagcaaaggcagtgag

SEQ ID NO: 89 - DNA, 32 bp, Artificial sequence
  derived from obi1
ctgccatggatcctcctataatttctttgcaa

SEQ ID NO: 90 - DNA, 30 bp, Artificial sequence
  derived from obi1
ctggaattctatggcgcagactgttcagaa

SEQ ID NO: 91 - DNA, 32 bp, Artificial sequence
  derived from obi1
ctgctcgagtcatggcttaaaggactttggag

SEQ ID NO: 92 - DNA, 40 bp, Artificial sequence
  derived from cdt1
aattctatgtatgatgttcctgattatgctagcctctaat

SEQ ID NO: 93 - DNA, 40 bp, Artificial sequence
  derived from cdt1
ctagattagaggctagcataatcaggaacatcatacatag

SEQ ID NO: 94 - DNA, 27 bp, Artificial sequence
  derived from cdt1
ctgaagcttaccatggagcagcgccgc

SEQ ID NO: 95 - DNA, 25 bp, Artificial Sequence
  derived from cdt1
ctggaattcagcccctcctcagcac

SEQ ID NO: 96 - DNA, 3552 bp, Homo sapiens; identical to SEQ ID NO: 31

SEQ ID NO: 97 - DNA, 2181 bp, Homo sapiens
atggctcagaccgtgcagaatgttacattgtcgctcactctgcccatcacgtgccacatttgcttggggaaggtacgtcagcctgtcatatgcatcaacaaccatgtattttgttcgatttgtattgatttgtggttgaagaataatagccagtgtccagcttgcagagtccccatcactcctgaaaatccttgcaaagaaattataggaggaacaagtgaaagtgaacctatgctaagccatacggtcaggaagcatcttcggaaaactagacttgaattactacacaaagaatatgaggacgaaatagattgtttacagaaagaagtagaagagcttaagagtaaaaatctcagcttggagtcacagatcaaaactattctggatcctttaaccttggtgcagggcaaccaaaatgaagacaaacatctagtcacagataatccaagtaaaattaacccagaaactgtagcagagtggaagaaaaaactcagaacagctaatgaaatctatgaaaaagtgaaagatgatgtggataagctaaaggaggcaaataaaaaattgaaattggaaaatggtggtctggtgagggagaatttacgactgaaggctgaagttgataacagatcacctcaaaagtttggaaggtttgcagttgctgctcttcagtccaaagtagaacagtatgagcgtgaaaccaatcgcctcaagaaagccctggaacgaagtgataagtatatagaggaactagaatctcaagttgcacagctaaaaaattcaagtgaagagaaagaagctatgaattccatttgccagacagcactttctgcagatggcaaagggagcaaaggcagtgaggaggatgtggtgtcaaagaatcaaggcgatagtgccagaaagcagcctggctcatccacctccagttcttctcacctagcgaagccttccagcagcagactgtgtgacaccagttctgcaaggcaggaaagtaccagcaaagcagaccttaactgttctaagaacaaagacctatatcaagaacaggtagaagtaatgttagatgtgacagatacaagtatggatacttatttggaaagagaatgggggaataaaccaagtgactgtgtaccctacaaagatgaagaactttatgatcttccagctccttgtactcctttgtcccttagttgccttcagctcagtactccagaaaatagagagagctctgtggtccaagcaggaggttccaaaaagcactcaaaccatctcagaaaattggtgtttgatgatttttgtgattcttcaaatgtttctaataaagattcttcagaagatgatataagtagaagtgaaaatgaaaagaaatcagaatgtttttcttccccaaagacaggattttgggactgttgttccacaagctatgcccaaaacttagattttgaaagttcagaggggaacacgatagcaaattctgttggagaaatatcttcaaaattgagtgagaaatcaggcttatgtttatccaaaaggttgaattctattcgctcttttgaaatgaaccggacaagaacatccagtgaagcatcgatggatgctgcttaccttgacaaaatctctgagttggattcaatgatgtcagagtcagacaacagcaagagcccttgtaataacggttttaagtcactggatttggatgggttatcaaagtcatctcaaggcagtgaatttcttgaggaacctgataagttggaagaaaaaactgagctaaacctttccaaaggttctctaactaatgatcagttagaaaatggaagtgaatggaaacccacttctttttttctcctctctccatctgaccaagaaatgaatgaagatttttcactccattccagttcttgtccagtaactaatgaaatcaaacccccaagctgcttgtttcagacagagttttcccagggcattttgttaagcagttcacatcgactatttgaagatcaaagatttgggtcatctttgtttaagatgtcctcagagatgcacagtcttcataaccaccttcagtctccttggtctacttcctttgtgcctgaaaagaggaataaaaatgtgaatcaatcaacaaaaagaaaaatccagagcagcctttccagtgccagcccatcaaaagcaactaaaagttga

SEQ ID NO: 98 - DNA, 3550 bp, Homo sapiens
ggcgaagagtagcggtaggtcggcgggacttccgtgttggcgggattctgaacgctgccatggctcagaccgtgcagaatgttacattgtcgctcactctgcccatcacgtgccacatttgcttggggaaggtacgtcagcctgtcatatgcatcaacaaccatgtattttgttcgatttgtattgatttgtggttgaagaataatagccagtgtccagcttgcagagtccccatcactcctgaaaatccttgcaaagaaattataggaggaacaagtgaaagtgaacctatgctaagccatacggtcaggaagcatcttcggaaaactagacttgaattactacacaaagaatatgaggacgaaatagattgtttacagaaagaagtagaagagcttaagagtaaaaatctcagcttggagtcacagatcaaaactattctggatcctttaaccttggtgcagggcaaccaaaatgaagacaaacatctagtcacagataatccaagtaaaattaacccagaaactgtagcagagtggaagaaaaaactcagaacagctaatgaaatctatgaaaaagtgaaagatgatgtggataagctaaaggaggcaaataaaaaattgaaattggaaaatggtggtctggtgagggagaatttacgactgaaggctgaagttgataacagatcacctcaaaagtttggaaggtttgcagttgctgctcttcagtccaaagtagaacagtatgagcgtgaaaccaatcgcctcaagaaagccctggaacgaagtgataagtatatagaggaactagaatctcaagttgcacagctaaaaaattcaagtgaagagaaagaagctatgaattccatttgccagacagcactttctgcagatggcaaagggagcaaaggcagtgaggaggatgtggtgtcaaagaatcaaggcgatagtgccagaaagcagcctggctcatccacctccagttcttctcacctagcgaagccttccagcagcagactgtgtgacaccagttctgcaaggcaggaaagtaccagcaaagcagaccttaactgttctaagaacaaagacctatatcaagaacaggtagaagtaatgttagatgtgacagatacaagtatggatacttatttggaaagagaatgggggaataaaccaagtgactgtgtaccctacaaagatgaagaactttatgatcttccagctccttgtactcctttgtcccttagttgccttcagctcagtactccagaaaatagagagagctctgtggtccaagcaggaggttccaaaaagcactcaaaccatctcagaaaattggtgtttgatgatttttgtgattcttcaaatgtttctaataaagattcttcagaagatgatataagtagaagtgaaaatgaaaagaaatcagtgtttttcttccccaaagacaggattttgggactgttgttccacaagctatgcccaaaacttagattttgaaagttcagaggggaacacgatagcaaattctgttggagaaatatcttcaaaattgagtgagaaatcaggcttatgtttatccaaaaggttgaattctattcgctcttttgaaatgaaccggacaagaacatccagtgaagcatcgatggatgctgcttaccttgacaaaatctctgagttggattcaatgatgtcagagtcagacaacagcaagagcccttgtaataacggttttaagtcactggatttggatgggttatcaaagtcatctcaaggcagtgaatttcttgaggaacctgataagttggaagaaaaaactgagctaaacctttccaaaggttctctaactaatgatcagttagaaaatggaagtgaatggaaacccacttctttttttctcctctctccatctgaccaagaaatgaatgaagatttttcactccattccagttcttgtccagtaactaatgaaatcaaacccccaagctgcttgtttcagacagagttttcccagggcattttgttaagcagttcacatcgactatttgaagatcaaagatttgggtcatctttgtttaagatgtcctcagagatgcacagtcttcataaccaccttcagtctccttggtctacttcctttgtgcctgaaaagaggaataaaaatgtgaatcaatcaacaaaaagaaaaatccagagcagcctttccagtgccagcccatcaaaagcaactaaaagttgactcattagaaaggtgtcatttgtggttttgtcctgagagaaatagaaaagttgttaaagttaccttttttcctcataaaagttctatacaaattggaattgataatctttagtcaagtatcaagtcaggatggtggattaacctgtacccagaatacttattgttcattttgaaaagactttgttcttttcatttttatttgggagtctttgtgaccagagaagttagggaggaggttatttttgtgttttggggttggttggttggttggttttgtttttggttttgtttttttactgaatttgatatgtatctcggttggatatacattgtttttttaaaaaatgttatttaactgttagatacagtggcctgttgataagccccacttgtcttcagaacttggatttcttaaataaaacttttagtgttgtctatacactgctcaataagacacttgagtttaagcttttcccagggtggaaattattttacctgtccctttttatttatgtttagtgatggcctagtttttctgcagggccatgatggagaaatagcactctagccttagtccaatattgatttactttctttttttaggttttatgtatatgtttgcattttttagcattgtgttttgtccagttttgtgaaaatgttctgctagtatgaaagaaaacattttctatatgaagacatttgttttatgttaggtagcttacattttctcctctgcgtgtgtgtgtatgtgtgtaaaatcagaaatttagcatactatggaaagaaggcatggagcacttgggtttagaggaacctaaaacatcatagcttcattgttccagatgtaacaggtttgaaagagctcatcgccaagttcttgatccacttgcattccaggggagttttcttttgagtagtatgtttcttgtttgcatgttcctgttctttgtggaaactatgcatggtagcatttttgcttgctgtgttttccatacttaagaaaaagaggtttcagttggctgatagaatatcttttatgtaggacaaaacttttctgtgaagagtgttgagggggtgaagataggtaagaggtaagcacaatttttaatttaggctctgaaaaagtgtattgttctaaacgtatttggtatgcctatataggtctttaaaaatgggtatgtatgctgtttaatgtgcactgaacattttacattaatattgtactgttttacattaatactgcatgcttttctatgtgaattgaataaagaatgtcataagcactgtgttcaaaaaaaaaaaaaaaaaa

SEQ ID NO: 99 - DNA, 1440 bp, Homo sapiens
atggctcagaccgtgcagaatgttacattgtcgctcactctgcccatcacgtgccacatttgcttggggaaggtacgtcagcctgtcatatgcatcaacaaccatgtattttgttcgatttgtattgatttgtggttgaagaataatagccagtgtccagcttgcagagtccccatcactcctgaaaatccttgcaaagaaattataggaggaacaagtgaaagtgaacctatgctaagccatacggtcaggaagcatcttcggaaaactagacttgaattactacacaaagaatatgaggacgaaatagattgtttacagaaagaagtagaagagcttaagagtaaaaatctcagcttggagtcacagatcaaaactattctggatcctttaaccttggtgcagggcaaccaaaatgaagacaaacatctagtcacagataatccaagtaaaattaacccagaaactgtagcagagtggaagaaaaaactcagaacagctaatgaaatctatgaaaaagtgaaagatgatgtggataagctaaaggaggcaaataaaaaattgaaattggaaaatggtggtctggtgagggagaatttacgactgaaggctgaagttgataacagatcacctcaaaagtttggaaggtttgcagttgctgctcttcagtccaaagtagaacagtatgagcgtgaaaccaatcgcctcaagaaagccctggaacgaagtgataagtatatagaggaactagaatctcaagttgcacagctaaaaaattcaagtgaagagaaagaagctatgaattccatttgccagacagcactttctgcagatggcaaagggagcaaaggcagtgaggaggatgtggtgtcaaagaatcaaggcgatagtgccagaaagcagcctggctcatccacctccagttcttctcacctagcgaagccttccagcagcagactgtgtgacaccagttctgcaaggcaggaaagtaccagcaaagcagaccttaactgttctaagaacaaagacctatatcaagaacaggtagaagtaatgttagatgtgacagatacaagtatggatacttatttggaaagagaatgggggaataaaccaagtgactgtgtaccctacaaagatgaagaactttatgatcttccagctccttgtactcctttgtcccttagttgccttcagctcagtactccagaaaatagagagagctctgtggtccaagcaggaggttccaaaaagcactcaaaccatctcagaaaattggtgtttgatgatttttgtgattcttcaaatgtttctaataaagattcttcagaagatgatataagtagaagtgaaaatgaaaagaaatcagtgtttttcttccccaaagacaggattttgggactgttgttccacaagctatgcccaaaacttagattttga

SEQ ID NO: 100 - protein, 479 aa, Homo sapiens
MAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALSADGKGSKGSEEDVVSKNQGDSARKQPGSSTSSSSHLAKPSSSRLCDTSSARQESTSKADLNCSKNKDLYQEQVEVMLDVTDTSMDTYLEREWGNKPSDCVPYKDEELYDLPAPCTPLSLSCLQLSTPENRESSVVQAGGSKKHSNHLRKLVFDDFCDSSNVSNKDSSEDDISRSENEKKSVFFFPKDRILGLLFHKLCPKLRF

SEQ ID NO: 101 - protein, 27 aa, Artificial Sequence
  derived from human Obi1 protein
KSECFSSPKTGFWDCCSTSYAQNLDFE

SEQ ID NO: 102 - DNA, 81 bp, Artificial Sequence
  derived from human Obi1 gene
aaatcagaatgtttttcttccccaaagacaggattttgggactgttgttccacaagctatgcccaaaacttagattttgaa

SEQ ID NO: 103 - DNA, 79 bp, Artificial Sequence
  derived from human mutated Obi1 gene
aaatcagtgtttttcttccccaaagacaggattttgggactgttgttccacaagctatgcccaaaacttagattttgaa

SEQ ID NO: 104 - protein, 25 aa, Artificial Sequence
  derived from mutated Obi1 protein
KSVFFFPKDRILGLLFHKLCPKLRF

SEQ ID NO: 105 - DNA, 1104 bp, Artificial Sequence
  ucliic acid coding for a dominant negative form of Obi1
atggagcaaaagctcatttctgaagaggacttgaatgaaatggagcaaaagctcatttctgaagaggacttgaatgaaatggagcaaaagctcatttctgaagaggacttgaatgaaatggagcaaaagctcatttctgaagaggacttgaatgaaatggagcaaaagctcatttctgaagaggacttgaatgaaatggagagcttgggcgacctcaccatgtctctggctctcaaactggctggactggacatcggatccatggctcagaccgtgcagaatgttacattgtcgctcactctgcccatcacgtgccacatttgcttggggaaggtacgtcagcctgtcatatgcatcaacaaccatgtattttgttcgatttgtattgatttgtggttgaagaataatagccagtgtccagcttgcagagtccccatcactcctgaaaatccttgcaaagaaattataggaggaacaagtgaaagtgaacctatgctaagccatacggtcaggaagcatcttcggaaaactagacttgaattactacacaaagaatatgaggacgaaatagattgtttacagaaagaagtagaagagcttaagagtaaaaatctcagcttggagtcacagatcaaaactattctggatcctttaaccttggtgcagggcaaccaaaatgaagacaaacatctagtcacagataatccaagtaaaattaacccagaaactgtagcagagtggaagaaaaaactcagaacagctaatgaaatctatgaaaaagtgaaagatgatgtggataagctaaaggaggcaaataaaaaattgaaattggaaaatggtggtctggtgagggagaatttacgactgaaggctgaagttgataacagatcacctcaaaagtttggaaggtttgcagttgctgctcttcagtccaaagtagaacagtatgagcgtgaaaccaatcgcctcaagaaagccctggaacgaagtgataagtatatagaggaactagaatctcaagttgcacagctaaaaaattcaagtgaagagaaagaagctatgaattccatttgccagacagcactttctgcagatggcaaataa

SEQ ID NO: 106 - protein, 367 aa, Artificial Sequence
  dominant negative form of OBI1
MEQKLISEEDLNEMEQKLISEEDLNEMEQKLISEEDLNEMEQKLISEEDLNEMEQKLISEEDLNEMESLGDLTMSLALKLAGLDIGSMAQTVQNVTLSLTLPITCHICLGKVRQPVICINNHVFCSICIDLWLKNNSQCPACRVPITPENPCKEIIGGTSESEPMLSHTVRKHLRKTRLELLHKEYEDEIDCLQKEVEELKSKNLSLESQIKTILDPLTLVQGNQNEDKHLVTDNPSKINPETVAEWKKKLRTANEIYEKVKDDVDKLKEANKKLKLENGGLVRENLRLKAEVDNRSPQKFGRFAVAALQSKVEQYERETNRLKKALERSDKYIEELESQVAQLKNSSEEKEAMNSICQTALSADGK

SEQ ID NO: 107 - DNA, 4352 bp, Artificial Sequence
  vector pCS2 comprising Myc tag
cgccattctgcctggggacgtcggagcaagcttgatttaggtgacactatagaatacaagctacttgttctttttgcaggatcccatcgatttaaagctatggagcaaaagctcatttctgaagaggacttgaatgaaatggagcaaaagctcatttctgaagaggacttgaatgaaatggagcaaaagctcatttctgaagaggacttgaatgaaatggagcaaaagctcatttctgaagaggacttgaatgaaatggagcaaaagctcatttctgaagaggacttgaatgaaatggagagcttgggcgacctcaccatggagcaaaagctcatttctgaagaggacttgaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 108 - DNA, 52 bp, Artificial Sequence
  derived from NES of PKI
catgtctctggctctcaaactggctggactggacatcggatccatggtcttg

SEQ ID NO: 109 - DNA, 52 bp, Artificial Sequence
  derived from NES of PKI
aattcaagaccatggatccgatgtccagtccagccagtttgagagccagaga

(19) United States
(12) Patent Application Publication
(10) Pub. No.: US 2016/0122742 A1
(43) Pub. Date: May 5, 2016

(54) PROTEIN INVOLVED IN DNA REPLICATION, AND MODULATION OF ITS ACTIVITY

(71) Applicants: Marcel MECHALI, Montferrier Sur Lez (FR); Coulombe PHILIPPE, Montpellier (FR)

(72) Inventors: Marcel MECHALI, Montferrier Sur Lez (FR); Philippe COULOMBE, Montpellier (FR)

(21) Appl. No.: 14/894,428

(22) PCT Filed: Jun. 2, 2014
(86) PCT No.: PCT/EP2014/061369

§ 371 Date: Nov. 27, 2015

(30) Foreign Application Priority Data
May 31, 2013 (EP) 13305725.7

(51) Int. Cl. C12N 9/00; A61K 38/53; C12N 15/113

(52) U.S. Cl. CPC C12N 9/93; C12N 15/1137; A61K 38/53; C12Y 603/02019; C12N 2320/30; C12N 2310/14

(57) ABSTRACT

A composition including at least a protein consisting of an amino acid sequence as set forth in SEQ ID NO: 1, or a salt or a solvate thereof, in association with a pharmaceutically acceptable carrier, for its use for treating pathologies link to DNA replication.

The present invention relates to a protein involved in DNA replication as drug. The invention also relates to compounds inhibiting the expression or the activity of said protein.

Chromosomal DNA replication in eukaryotic cells is highly complicated and sophisticatedly regulated. Owing to its large size, a typical eukaryotic genome contains hundreds to tens of thousands of initiation sites called DNA replication origins where DNA synthesis takes place. Multiple initiation sites remove the constraint of a genome size because only a certain amount of DNA can be replicated from a single origin in a limited time. The activation of these multiple origins must be coordinated so that each segment of chromosomal DNA is precisely duplicated only once per cell cycle. Although DNA replication is a vital process for cell growth and its mechanism is highly conserved, recent studies also reveal significant diversity in origin structure, assembly of pre-replication complex (pre-RC) and regulation of replication initiation along evolutionary lines.

It is crucial to maintain genomic integrity in eukaryotic cells. During DNA replication, errors in pre-RC assembly often result in substantial changes in the amount of genetic material, which will cause cell death, or transform cells to become malignant tumors.

### The ORC Proteins

The first origin recognition complex (ORC) proteins to be identified were purified from cell extracts as a heterohexameric complex that specifically binds to origins of DNA replication and the subunits were named Orc1 through Orc6. Subsequently, orthologs of ORC1-ORC5 were identified in organisms as diverse as Drosophila melanogaster, Arabidopsis thaliana and Homo sapiens, strongly suggesting that these genes are likely to exist in all eukaryotes.

The ORC needs to be in an ATP-bound state so that it can interact with Cdc6p creating events that will produce the unwinding of the double stranded DNA which will promote the hydrolysis of the ATP by the ORC. Once the ORC is bound to the origin, the complex is retained in an ATP-bound state and the ATP hydrolysis is reserved for a downstream step in initiation. When the ORC binds to DNA at replication origin sites in an ATP-dependent manner it then serves as a scaffold for the assembly of other key initiation factors of the pre-replicative complex (pre-RC) which include Cdc6, Cdt1, and Mcm proteins.

Recently, mutations in genes encoding ORC1, ORC4, ORC6, CDT1, and CDC6 were identified in patients displaying Seckel syndrome (SS) and/or Meier-Gorlin syndrome (MGS) [Bicknell L S et al. (2011) Nat Genet 43: 356-359; Bicknell L S, et al. (2011) Nat Genet 43: 350-355; Guernsey D L, et al. (2011) Nat Genet 43: 360-364].

Seckel syndrome, Majewski osteodysplastic primordial dwarfism (MOPD) type II and Meier-Gorlin syndrome represent three disorders which share overlapping clinical features that include pronounced microcephaly, severe intrauterine growth retardation and post natal growth delay.

However, even if these mutations are associated with some of patients afflicted by these syndromes, some orphan individuals do not harbor any mutations or expression abnormality in the genes encoding ORC1, ORC4, ORC6, CDT1, and CDC6.

Therefore, there is a need to identify other genes in which mutations could be responsible of the syndromes developed by the above orphan individuals.

### One Aim of the Invention is to Identify Such Gene.

Another aim of the invention is to identify proteins that could be responsible of the above syndromes, or other syndrome linked to defects in unknown protein controlling DNA replication.

Still another aim of the invention is to provide a new efficient new medicine intended for the treatment of the above syndromes.

The invention relates to a composition comprising at least one of the following compounds:
a protein consisting of an amino acid sequence as set forth in SEQ ID NO: 1, or a fragment thereof provided that said fragment retains properties to interact with the origin recognition complex, or eventually retains oncogenic properties or dimerization properties,
a protein derived from the protein as set forth in SEQ ID:1 by substitution, deletion, or insertion of at least one nucleotide provided that this protein retains ubiquitin ligase activity and/or participate to DNA replication,
a nucleic acid molecule coding for said proteins or said fragments,
a compound affecting, i.e. inhibiting or activating, the expression or the activity of said protein,
   a salt or a solvate thereof,
   in association with a pharmaceutically acceptable carrier.

The invention is based on the characterization by the inventors of a new protein that physically interacts with the ORC protein complex and that is involved in DNA replication.

By a purification process of the ORC protein complex, the inventors have identified a new ORC protein complex partner which has been identified to specifically interact with both ORC1 and LRWD1 proteins. The process for purifying the protein is described hereafter.

The ORC1 protein is a component of the origin recognition complex (ORC) that binds origins of replication. The DNA-binding of ORC1 is ATP-dependent.

The LRW1 protein is an associated component of the ORC required to recruit and stabilize the ORC complex to chromatin during G1 to establish pre-replication complex (preRC) and to heterochromatic sites in post-replicated cells. This protein binds a combination of DNA and histone methylation repressive marks on heterochromatin: it binds histone H3 and H4 trimethylation marks H3K9me3, H3K27me3 and H4K20me3 in a cooperative manner with DNA methylation.

The inventors have identified that the protein disclosed in databases as the RNF219 protein specifically interacts with only the above proteins (ORC1 and LRWD1) and is binding the ORC complex.

In view of the characterization of the interaction, RNF219 is now named: ORC Ubiquitin-ligase 1 or OBI1).

The human OBI1 protein consists of the amino acid sequence as set forth in SEQ ID NO: 1.

The composition according to the invention can therefore contain, as active substance, the OBI1 protein as set forth in SEQ ID NO: 1.

The invention also encompasses a protein derived from the protein as set forth in SEQ ID:1, said protein consisting of the amino acid sequence as set forth in SEQ ID NO: 1 and having at least one substitution and/or one deletion and/or one insertion of at least one nucleotide provided that said protein retains ubiquitin ligase activity and/or participates to DNA replication.

The invention encompasses also a composition as defined above, comprising as active substance a protein having at least 20% identity with SEQ ID NO: 1, in particular a protein having at least 20% identity with SEQ ID NO: 1 and containing the amino acid sequence SEQ ID NO: 28. SEQ ID NO: 28 represents the amino acid sequence of the zinc finger/ring finger of the OBI1 proteins.

Thus, in the invention, a protein having at least 20% identity with SEQ ID NO: 1 defines a proteins having 20% identity, 21% identity, 22% identity, 23% identity, 24% identity, 25% identity, 26% identity, 27% identity, 28% identity, 29% identity, 30% identity, 31% identity, 32% identity, 33% identity, 34% identity, 35% identity, 36% identity, 37% identity, 38% identity, 39% identity, 40% identity, 41% identity, 42% identity, 43% identity, 44% identity, 45% identity, 46% identity, 47% identity, 48% identity, 49% identity, 50% identity, 51% identity, 52% identity, 53% identity, 54% identity, 55% identity, 56% identity, 57% identity, 58% identity, 59% identity, 60% identity, 61% identity, 62% identity, 63% identity, 64% identity, 65% identity, 66% identity, 67% identity, 68% identity, 69% identity, 70% identity, 71% identity, 72% identity, 73% identity, 74% identity, 75% identity, 76% identity, 77% identity, 78% identity, 79% identity, 80% identity, 81% identity, 82% identity, 83% identity, 84% identity, 85% identity, 86% identity, 87% identity, 88% identity, 89% identity, 90% identity, 91% identity, 92% identity, 93% identity, 94% identity, 95% identity, 96% identity, 97% identity, 98% identity or 99% identity with SEQ ID NO: 1. Such proteins are, for instance, the proteins consisting of the amino acid sequence as set forth in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 or SEQ ID NO: 27.

All these proteins represent the OBI1 proteins of different eukaryotic species. The above lists are not limitative, and the skilled person is able to determine any new Obi1 protein.

The invention also encompasses a composition comprising fragments of the above proteins, fragments retaining at least one of the function of the protein: i.e. said fragments retaining at least an oncogenic function, an ubiquitin ligase function or a dimerization function.

The advantageous fragments contained in the composition according to the invention are:
the peptide consisting of the amino acid sequence SEQ ID NO: 59,
the peptide consisting of the amino acid sequence SEQ ID NO: 29,
the peptide consisting of the amino acid sequence SEQ ID NO: 30,
the peptide consisting of the amino acid sequence SEQ ID NO: 60,
the peptide consisting of the amino acid sequence SEQ ID NO: 61,
the peptide consisting of the amino acid sequence SEQ ID NO: 62,
the peptide consisting of the amino acid sequence SEQ ID NO: 63, and
the peptide consisting of the amino acid sequence SEQ ID NO: 100.

All the proteins and fragments as defined above can also be tagged with specific tags, in order to enhance and/or facilitate their purification. The tags can be located at the N-terminus part or at the C-terminus part of the proteins or fragments. In some advantageous embodiments, the proteins can be tagged at both their N- and C-terminus parts, such protein being advantageously tagged with two different tags.

The skilled person is able, with his common knowledge, to determine the most appropriated tags. The following list, which not intend to restrict the scope of the invention, represents the common tags that can be used: GFP, EGFP, YFP, CFP, dsRed, Myc tag, E tag, FLAG tag, Glu-Glu tag, GST tag, HA tag, His tag, in particular 6×HIS, HSV tag, luciferase, MBP, protein C tag, S tag, T7 tag, V5 tag, VSV-g tag, avidin/streptavidin/strep tag, thioredoxin, His-patch thioredoxin, 6-galactosidase, chloramphenicol acetyltransferase, cellulose binding domains (CBDs), chitin binding domain, staphylococcal protein A, streptococcal protein G, neo, hyg, pac, zeo, gpt, ble, dhfr, hpt and npt II.

It is also possible to use in the protein according to the invention nuclear sequences such as Nuclear Export Sequence (NES) or Nuclear Localization Signal (NLS), in order to specifically address respectively protein comprising them to the cytoplasm or the nucleus.

The invention also encompasses a composition comprising nucleic acid molecules coding for the proteins or the fragments as defined above.

In the invention the nucleic acid coding for the protein consisting of SEQ ID NO: 1 consists of the mRNA nucleic sequence as set forth in SEQ ID NO: 31.

The composition as defined above may also contain inhibitors of the expression or of the activity of the above mentioned protein.

In the invention salt(s), as used herein, means those salts of compounds of the invention that are safe and effective for use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, oxalate, maleate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate (i.e., 1,1′-methylene-bis-(2-hydroxy-3-naphthoate)), mesylate, besylate, tosylate and tartrate salts. Suitable base salts include, but are not limited to, aluminum, calcium, lithium, magnesium, potassium, sodium, zinc, and diethanolamine salts.

Advantageously, the invention relates to a composition as defined above, wherein said nucleic acid molecule comprises the nucleic acid sequence SEQ ID NO: 31, or a nucleic acid sequence having at least 20% identity, i.e. 20% identity, 21% identity, 22% identity, 23% identity, 24% identity, 25% identity, 26% identity, 27% identity, 28% identity, 29% identity, 30% identity, 31% identity, 32% identity, 33% identity, 34% identity, 35% identity, 36% identity, 37% identity, 38% identity, 39% identity, 40% identity, 41% identity, 42% identity, 43% identity, 44% identity, 45% identity, 46% identity, 47% identity, 48% identity, 49% identity, 50% identity, 51% identity, 52% identity, 53% identity, 54% identity, 55% identity, 56% identity, 57% identity, 58% identity, 59% identity, 60% identity, 61% identity, 62% identity, 63% identity, 64% identity, 65% identity, 66% identity, 67% identity, 68% identity, 69% identity, 70% identity, 71% identity, 72% identity, 73% identity, 74% identity, 75% identity, 76% identity, 77% identity, 78% identity, 79% identity, 80% identity, 81% identity, 82% identity, 83% identity, 84% identity, 85% identity, 86% identity, 87% identity, 88% identity, 89% identity, 90% identity, 91% identity, 92% identity, 93% identity, 94% identity, 95% identity, 96% identity, 97% identity, 98% identity or 99% identity, with the nucleic acid sequence SEQ ID NO: 31, provided that he protein coded by said nucleic acid sequences retains ubiquitin ligase activity, and/or participates to DNA replication.

In one another advantageous embodiment, the invention relates to a composition previously defined, wherein said nucleic acid molecule is contained in a vector, said vector comprising means allowing the expression of said nucleic acid molecule.

In the invention the vector is advantageously an expression vector which is a plasmid or virus designed for protein expression in cells. The vector is used to introduce a specific gene into a target cell, and can command the cell mechanism for protein synthesis to produce the protein encoded by the gene.

The vector is engineered to contain regulatory sequences that act as enhancer and promoter regions and lead to efficient transcription of the gene carried on the expression vector.

An expression vector have elements necessary for protein expression that include a strong promoter, the correct translation initiation sequence such as a ribosomal binding site and start codon, a strong termination codon, and a transcription termination sequence. There are differences in the machinery for protein synthesis between prokaryotes and eukaryotes, therefore the expression vectors must have the elements for expression that is appropriate for the chosen host. For example, prokaryotes expression vectors would have a Shine-Dalgarno sequence at its translation initiation site for the binding of ribosomes, while eukaryotes expression vectors contains the Kozak consensus sequence.

Vector may also comprise replication origin allowing the replication of the vector in bacteria. Furthermore, the vector as defined above may contain at least one selection marker allowing selection of transformed cells. These markers are well known in the art and the skilled person is able to determine which marker is more appropriate for a determined experiment.

In still another advantageous embodiment, the invention relates to a composition as previously defined wherein said compound inhibiting the expression of the protein consisting of an amino acid sequence as set forth in SEQ ID NO: 1, or the protein derived from the protein as set forth in SEQ ID:1 by substitution, deletion, or insertion of at least one nucleotide provided that this protein retains ubiquitin ligase activity and/or participate to DNA replication, in particular a protein having at least 20% identity with SEQ ID NO: 1 and containing the amino acid sequence SEQ ID NO: 28, as defined above, is chosen among a siRNA, miRNA, shRNA, RNA antisense, DNA antisense, antibodies and a fragment of the protein consisting of an amino acid sequence as set forth in SEQ ID NO: 1, said fragment comprising the amino acid sequence SEQ ID NO: 29, SEQ ID NO 30, SEQ ID NO: 59, SEQ ID NO: 60, SEQ ID NO: 61, SEQ ID NO: 62, SEQ ID NO: 63, SEQ ID NO: 100 or SEQ ID NO: 106.

siRNA, shRNA or miRNA according to the invention inhibit OBI1 gene expression by RNA interference mechanism.

RNA interference is a highly conserved biological mechanism inducing specific repression of genes by specifically destroying mRNA, or inhibition translation of said RNA.

In 1998, Fire et al [Fire et al., Nature. 1998 Feb. 19; 391(6669):806-11] demonstrated that a double-stranded is produced in cells by a Class III RNA endonuclease, the DICER complex, and small inhibiting double-stranded RNA (siRNA) of about 19 to 28 nucleotides are produced.

Incorporated to the enzymatic <<RNA-Induced Silencing Complex>> RISC complex, said siRNA are deshybridized and can therefore hybridize with the complementary sequence contained in mRNA. The “captured” mRNA is then destroyed, or its translation by ribosomal particles is inhibited.

Small hairpin RiboNucleic Acid—shRNA are double-stranded molecules comprising both the sense and the antisense strand of a siRNA, said sense and antisense strands being linked by a linker. These molecules form a hairpin, and the linker is eliminated to allow the liberation of a siRNA.

Micro RNA—miRNA are encoded by eukaryotic nuclear DNA and act as si- or mi-RNA.

The invention also relates to the composition as defined above, for its use as medicine. The invention further relates to the composition as defined above, for its use for the treatment or the prevention of a human or animal pathology linked to a dysfunction of the OBI1 gene or to the DNA replication.

In other words, the invention relates to a composition comprising at least one of the following compounds:
a protein consisting of an amino acid sequence as set forth in SEQ ID NO: 1, or a fragment thereof provided that said fragment retains properties to interact with the origin recognition complex,
a protein derived from the protein as set forth in SEQ ID NO:1 by substitution of at least one nucleotide provided that said protein retains ubiquitin ligase activity and/or participates to DNA replication and/or interacts with ORC complex, and/or retaining its ability to self-interact, i.e. to form a homodimere or to an heterodimere with the full length Obi1 protein,
a nucleic acid molecule coding for said proteins or said fragments,
a compound affecting the expression or the activity of said protein, a salt or a solvate thereof,

possibly in association with a pharmaceutically acceptable carrier, as defined above

for its use for the treatment or the prevention of a human or animal pathology linked to a dysfunction of the OBI1 gene as set forth in SEQ ID NO: 31 or the Obi1 protein as set forth in SEQ ID NO: 1, or the DNA replication.

The invention encompasses both:
the composition as defined above for its use for the treatment or the prevention of a human or animal pathology linked to a dysfunction of the OBI1 gene, or OBI1 protein, and
the composition as defined above for its use for the treatment or the prevention of a human or animal pathology linked to DNA replication.

The invention relates also to a method for treating human or animal pathology linked to a dysfunction of the OBI1 gene or linked to the DNA replication, said method comprising the administration of an effective amount, in a patient in a need thereof, of at least one of the following compounds:
a protein consisting of an amino acid sequence as set forth in SEQ ID NO: 1, or a fragment thereof provided that said fragment retains properties to interact with the origin recognition complex, or eventually retains oncogenic properties or dimerization properties,
a protein derived from the protein as set forth in SEQ ID:1 by substitution, deletion, or insertion of at least one nucleotide provided that this protein retains ubiquitin ligase activity and/or participate to DNA replication,
a nucleic acid molecule coding for said proteins or said fragments, and
a compound inhibiting the expression or the activity of said protein,
   or a salt or a solvate thereof.

Advantageously, the invention relates to the composition for its use according to the previous definition, wherein said pathology is a neoplastic disease or cancer, and wherein said composition comprises a compound inhibiting the expression of said nucleic acid molecule or the activity of said protein.

The compounds inhibiting the expression of the protein, i.e. the Obi1 protein, can be for instance the molecule inhibiting the expression by RNA interference. Such compounds are for instance those described hereafter.

The compounds inhibiting the activity of the protein, i.e. the Obi1 protein, are for instance compounds inhibiting the ubiquitin ligase activity of said protein.

Examples of such compounds are those discloses in Ceccarelli et al 2011 Cell 145:1075-87.

In one another advantageous embodiment, the invention relates to the composition for its use previously defined, wherein said pathology is a pathology linked to DNA replication is chosen among Meier-Gorlin syndrome, Seckel syndrome and Majewski osteodysplastic primordial dwarfism and wherein said composition comprises:
a protein consisting of an amino acid sequence as set forth in SEQ ID NO: 1, or a fragment thereof provided that said fragment retains properties to interact with the origin recognition complex, or
protein derived from the protein as set forth in SEQ ID:1 by substitution, deletion, or insertion of at least one nucleotide provided that this protein retains ubiquitin ligase activity and/or participate to DNA replication, or
a nucleic acid molecule coding for said proteins or said fragments.

The invention also relates to a nucleic acid molecule inhibiting the expression of the OBI1 gene, as defined above by RNA interference, said a nucleic acid molecule comprising or consisting essentially of, or consisting of one of the following sequences:

(SEQ ID NO: 32)
5′-GTAGAAGTAATGTTAGATG-3′,
(SEQ ID NO: 33)
5′-CCAAAGGTTCTCTAACTAA-3′,
(SEQ ID NO: 34)
5′-GGATTTGGATGGGTTATCA-3′,
(SEQ ID NO: 35)
5′-GAACGAAGTGATAAGTATA-3′,
(SEQ ID NO: 36)
5′-GUAGAAGUAAUGUUAGAUG-3′,
(SEQ ID NO: 37)
5′-CCAAAGGUUCUCUAACUAA-3′,
(SEQ ID NO: 38)
5′-GGAUUUGGAUGGGUUAUCA-3′,
(SEQ ID NO: 39)
5′-GAACGAAGUGAUAAGUAUA-3′,
(SEQ ID NO: 40)
5′-CATCTAACATTACTTCTAC-3′,
(SEQ ID NO: 41)
5′-TTAGTTAGAGAACCTTTGG-3′,
(SEQ ID NO: 42)
5′-TGATAACCCATCCAAATCC-3′,
(SEQ ID NO: 43)
5′-TATACTTATCACTTCGTTC-3′,
(SEQ ID NO: 44)
5′-CAUCUAACAUUACUUCUAC-3′,
(SEQ ID NO: 45)
5′-UUAGUUAGAGAACCUUUGG-3′,
(SEQ ID NO: 46)
5′-UGAUAACCCAUCCAAAUCC-3′,
and
(SEQ ID NO: 47)
5′-UAUACUUAUCACUUCGUUC-3′.

The invention also relates to a composition, in particular for its use as defined above, comprising at least one of the nucleic acid molecules as defined above.

The complementary sequence of the nucleic acid molecule comprising or consisting of SEQ ID NO: 32 comprises or consists of the sequence SEQ ID NO: 40. The complementary sequence of the nucleic acid molecule comprising or consisting of SEQ ID NO: 33 comprises or consists of the sequence SEQ ID NO: 41. The complementary sequence of the nucleic acid molecule comprising or consisting of SEQ ID NO: 34 comprises or consists of the sequence SEQ ID NO: 42. The complementary sequence of the nucleic acid molecule comprising or consisting of SEQ ID NO: 35 comprises or consists of the sequence SEQ ID NO: 43. The complementary sequence of the nucleic acid molecule comprising or consisting of SEQ ID NO: 36 comprises or consists of the sequence SEQ ID NO: 44. The complementary sequence of the nucleic acid molecule comprising or consisting of SEQ ID NO: 37 comprises or consists of the sequence SEQ ID NO: 45. The complementary sequence of the nucleic acid molecule comprising or consisting of SEQ ID NO: 38 comprises or consists of the sequence SEQ ID NO: 46. The complementary sequence of the nucleic acid molecule comprising or consisting of SEQ ID NO: 39 comprises or consists of the sequence SEQ ID NO: 47.

Thus, most advantageous siRNA according to the invention are one of the following siRNA:
siRNA comprising a sense strand comprising or consisting in SEQ ID NO: 36 and its complementary sequence, or antisense strand, comprising or consisting of SEQ ID NO: 44,
siRNA comprising a sense strand comprising or consisting in SEQ ID NO: 37 and its complementary sequence, or antisense strand, comprising or consisting of SEQ ID NO: 45,
siRNA comprising a sense strand comprising or consisting in SEQ ID NO: 38 and its complementary sequence, or antisense strand, comprising or consisting of SEQ ID NO: 46, and
siRNA comprising a sense strand comprising or consisting in SEQ ID NO: 39 and its complementary sequence, or antisense strand, comprising or consisting of SEQ ID NO: 47.

The above siRNA can also be modified by addition of compounds stabilizing siRNA structure.

For instance, the above siRNA may contain, in their 3′-end, a dinucleotide: a dithymidine (TT). Therefore, the siRNA according to the invention comprise one of the following sequences:

(SEQ ID NO: 48)
5′-GUAGAAGUAAUGUUAGAUGTT-3′,
(SEQ ID NO: 49)
5′-CCAAAGGUUCUCUAACUAATT-3′,
(SEQ ID NO: 50)
5′-GGAUUUGGAUGGGUUAUCATT-3′,
(SEQ ID NO: 51)
5′-GAACGAAGUGAUAAGUAUATT-3′,
(SEQ ID NO: 52)
5′-CAUCUAACAUUACUUCUACTT-3′,
(SEQ ID NO: 53)
5′-UUAGUUAGAGAACCUUUGGTT-3′,
(SEQ ID NO: 54)
5′-UGAUAACCCAUCCAAAUCCTT-3′,
and
(SEQ ID NO: 55)
5′-UAUACUUAUCACUUCGUUCTT-3′.

Thus, most advantageous siRNA according to the invention are one of the following siRNA:
siRNA comprising a sense strand comprising or consisting in SEQ ID NO: 48 and its complementary sequence, or antisense strand, comprising or consisting of SEQ ID NO: 52,
siRNA comprising a sense strand comprising or consisting in SEQ ID NO: 49 and its complementary sequence, or antisense strand, comprising or consisting of SEQ ID NO: 53,
siRNA comprising a sense strand comprising or consisting in SEQ ID NO: 50 and its complementary sequence, or antisense strand, comprising or consisting of SEQ ID NO: 54, and
siRNA comprising a sense strand comprising or consisting in SEQ ID NO: 51 and its complementary sequence, or antisense strand, comprising or consisting of SEQ ID NO: 55.

In one another advantageous embodiment, the invention relates to a product used as mentioned above, wherein said shRNA comprises or consists of one of the following nucleic acid molecules:
a nucleic acid molecule comprising or being constituted by the sequence SEQ ID NO: 3 followed by the sequence SEQ ID NO: 48, the 3′-end of SEQ ID NO:3 being linked to the 5′-end of SEQ ID NO: 52 by a linker,
a nucleic acid molecule comprising or being constituted by the sequence SEQ ID NO: 5 followed by the sequence SEQ ID NO: 49, the 3′-end of SEQ ID NO:5 being linked to the 5′-end of SEQ ID NO: 53 by a linker,
a nucleic acid molecule comprising or being constituted by the sequence SEQ ID NO: 7 followed by the sequence SEQ ID NO: 50, the 3′-end of SEQ ID NO:7 being linked to the 5′-end of SEQ ID NO: 54 by a linker, and
a nucleic acid molecule comprising or being constituted by the sequence SEQ ID NO: 9 followed by the sequence SEQ ID NO: 51, the 3′-end of SEQ ID NO:9 being linked to the 5′-end of SEQ ID NO: 55 by a linker.

The linker according to the invention can be chosen among the following linkers:

### 1) UUCAAGAGA (Brummelkamp, T. R., 2002 Science. 296(5567):550-3),

### 2) AAGUUCUCU (Promega),

### 3) UUUGUGUAG (Scherr, M., Curr Med Chem. 2003 February; 10(3):245-56.),

### 4) CUUCCUGUCA (SEQ ID NO: 56) (Schwarz D. S., 2003 Cell. 115(2):199-208.), and

### 5) CUCGAG.

The compounds inhibiting the activity of the protein, i.e. the Obi1 protein, can be, for instance, the protein fragments interfering with the activity of the wild type Obi1 protein. Such fragments are for instance those as set forth in SEQ ID NO: 29, SEQ ID NO 30, SEQ ID NO: 59, SEQ ID NO: 60, SEQ ID NO: 61, SEQ ID NO: 62, SEQ ID NO: 63, SEQ ID NO: 100 or SEQ ID NO: 106, in particular the fragments as set forth in SEQ ID NO: 29, SEQ ID NO: 61, SEQ ID NO: 100 and SEQ ID NO: 106.

The invention also relates to an inhibitor of the activity of the protein consisting of an amino acid sequence as set forth in SEQ ID NO: 1, or a protein derived from the protein as set forth in SEQ ID:1 by substitution, deletion, or insertion of at least one nucleotide provided that said protein retains ubiquitin ligase activity and/or participates to DNA replication, as defined above, characterized in that it comprises the amino acid sequence: SEQ ID NO: 29, SEQ ID NO 30, SEQ ID NO: 59, SEQ ID NO: 60, SEQ ID NO: 61, SEQ ID NO: 62, SEQ ID NO: 63, SEQ ID NO: 100 or SEQ ID NO: 106.

The above fragments may be used as purified proteins, in particular in association with an appropriate salt, or in a form of nucleic acid coding said fragment, possibly contained in a vector (viral vector), possibly in the vector comprising a Myc tag, and possibly derived from the vector as set forth in SEQ ID NO: 107.

These proteins, or fragments, and their nucleic acids are artificial, non naturally occurring, or purified from their natural context.

The invention further relates to the use of the protein consisting of an amino acid sequence as set forth in SEQ ID NO: 1, or protein derived from the protein as set forth in SEQ ID:1 by substitution, deletion, or insertion of at least one nucleotide provided that said protein retains ubiquitin ligase activity and/or participates to DNA replication, as defined above, or a fragment thereof as defined above as Lys6 ubiquitine ligase enzyme.

The invention also relates to a method for ubiquitinate at least one protein, in particular ORC3 and ORC5 subunits of the ORC complex, comprising a step of contacting said protein with the protein consisting of an amino acid sequence as set forth in SEQ ID NO: 1, protein derived from the protein as set forth in SEQ ID:1 by substitution, deletion, or insertion of at least one nucleotide provided that said protein retains ubiquitin ligase activity and/or participates to DNA replication, or a fragment thereof as defined above,

said protein comprising a chain of Lys6-linked poly ubiquitin.

The RING ligases that have been characterized to date yield different patterns of substrate ubiquitination.

RING domains recruit E2 enzymes that are thioesterified with ubiquitin and also activate E2 to discharge its ubiquitin cargo to a substrate.

A number of RING ligases primarily monoubiquitinate or oligoubiquitinate their substrates. Ubiquitination may terminate after the transfer of only one or a few ubiquitins because the substrate simply falls off the E3 before the reaction can proceed further. However, oligoubiquitination is specified by an internal ubiquitin-binding domain that caps the growing chain and prevents its further extension. Yet other E3-E2 pairs transfer multiple ubiquitins to substrate, but in the form of several monoubiquitins rather than a polyubiquitin chain.

Multiple monoubiquitination can directly promote degradation of cyclin B in vitro but most often probably does not function as a proteasome-targeting signal in vivo, as exemplified by modification and lysosomal targeting of the EGF receptor by c-Cbl. Other RING-E2 complexes transfer multiple ubiquitins to substrate preferentially in the form of a polyubiquitin chain.

RING-E2 complexes can assemble chains linked exclusively via the Lys6, Lys48, and Lys63 residues of ubiquitin. Ultimately, all seven lysine residues of ubiquitin (Lys6, Lys11, Lys27, Lys29, Lys33, Lys48, Lys63) can form chain linkages in vivo. Thus, the number of topological isomers is potentially huge if chains of mixed linkage are produced.

These modifications have a range of biological effects, from proteasome-dependent proteolysis (Lys48- and Lys11-linked polyubiquitin) to posttranslational control of protein function, structure, assembly, and/or localization (Lys63 and other linkages). The inventors have demonstrated that OBI1 protein is an E3 ubiquitin ligase that essentially forms Lys6 polyubiquitin chains (see example section).

The invention also relates to the use of an inhibitor of the protein consisting of an amino acid sequence as set forth in SEQ ID NO: 1, protein derived from the protein as set forth in SEQ ID:1 by substitution, deletion, or insertion of at least one nucleotide provided that said protein retains ubiquitin ligase activity and/or participates to DNA replication, as defined above, for inhibiting cell proliferation.

The inventors have demonstrated that OBI1 gene is an oncogene, as set forth in the example section.

Therefore, the inventors propose, and have tested, inhibitors of the expression, and/or activity of OBI1 in order to abolish its oncogenic function and in order to inhibit deregulated cell proliferation.

The invention also encompasses a composition comprising at least one inhibitor of the protein consisting of an amino acid sequence as set forth in SEQ ID NO: 1, or protein derived from the protein as set forth in SEQ ID:1 by substitution, deletion, or insertion of at least one nucleotide provided that said protein retains ubiquitin ligase activity and/or participates to DNA replication, as defined above, for its use for treating cancer. The invention relates also to a method for treating cancer comprising the administration of an effective amount, to a patient in a need thereof, of at least one inhibitor of the protein consisting of an amino acid sequence as set forth in SEQ ID NO: 1, or protein derived from the protein as set forth in SEQ ID:1 by substitution, deletion, or insertion of at least one nucleotide provided that said protein retains ubiquitin ligase activity and/or participates to DNA replication, as defined above.

The invention further relate to a process for purifying a protein consisting of the amino acid sequence SEQ ID NO: 1, or protein derived from the protein as set forth in SEQ ID:1 by substitution, deletion, or insertion of at least one nucleotide provided that said protein retains ubiquitin ligase activity and/or participates to DNA replication, as defined above, said process comprising:
a step of purifying either the ORC1 protein or the LRWD1 protein, and
a step of isolating the OBI1 protein.

In the invention, the ORC1 and LRWD1 proteins consist respectively of the amino acid sequence as set forth in SEQ ID NO: 57 and 58.

The invention further relate to a process for purifying a protein consisting of the amino acid sequence SEQ ID NO: 1, or protein derived from the protein as set forth in SEQ ID:1 by substitution, deletion, or insertion of at least one nucleotide provided that said protein retains ubiquitin ligase activity and/or participates to DNA replication, as defined above, said process comprising the steps of:
immunoprecipitating with a first specific antibody either the ORC1 protein or the LRWD1 protein,
eluting the purified proteins with a peptide which is specific of said first antibody,
immunoprecipitating with a second antibody specific antibody either the ORC1 protein or the LRWD1 protein,
eluting the purified proteins with a peptide which is specific of said second antibody, and
optionally further purifying the protein consisting of the amino acid sequence SEQ ID NO: 1, or protein derived from the protein as set forth in SEQ ID:1 by substitution, deletion, or insertion of at least one nucleotide provided that said protein retains ubiquitin ligase activity and/or participates to DNA replication, with a third specific antibody.

In one other advantageous embodiment, the invention relates to a process for purifying a protein consisting of the amino acid sequence SEQ ID NO: 1, or protein derived from the protein as set forth in SEQ ID:1 by substitution, deletion, or insertion of at least one nucleotide provided that said protein retains ubiquitin ligase activity and/or participates to DNA replication, said process comprising the steps of:
purifying a tagged ORC1 protein or a tagged LRWD1 protein, said tagged ORC and LRWD1 proteins being tagged with two different tags, said purification step being carried out by using s first tag,
eluting the purified proteins with the free tag used to purify said proteins,
purifying a tagged ORC1 protein or a tagged LRWD1 protein obtained from the first step by using the second tag,
eluting the purified proteins with the free tag used to purify said proteins,
isolating the OBI1 protein.

The above method is well known in the art as TAP-TAG purification process as discloses in Puig et al. METHODS 24, 218-229 (2001).

Tandem affinity purification (TAP) is a technique for studying protein-protein interactions. It involves creating a fusion protein with a designed piece, the TAP tag, on the end.

In the original version of the technique, the protein of interest with the TAP tag first binds to beads coated with IgG, the TAP tag is then broken apart by an enzyme, and finally a different part of the TAP tag binds reversibly to beads of a different type. After the protein of interest has been washed through two affinity columns, it can be examined for binding partners.

The original TAP method involves the fusion of the TAP tag to the C-terminus of the protein under study. The TAP tag consists of calmodulin binding peptide (CBP) from the N-terminal, followed by tobacco etch virus protease (TEV protease) cleavage site and Protein A, which binds tightly to IgG. The relative order of the modules of the tag is important because Protein A needs to be at the extreme end of the fusion protein so that the entire complex can be retrieved using an IgG matrix.

Once the fusion protein is translated within the host, the new protein at one end of the fusion protein would be able to interact with other proteins. Subsequently, the fusion protein is retrieved from the host by breaking the cells and retrieving the fusion protein through affinity selection, together with the other constituents attached to the new protein, by means of an IgG matrix.

After washing, TEV protease is introduced to elute the bound material at the TEV protease cleavage site. This eluate is then incubated with calmodulin-coated beads in the presence of calcium. This second affinity step is required to remove the TEV protease as well as traces of contaminants remaining after the first affinity step.[2] After washing, the eluate is then released with ethylene glycol tetraacetic acid (EGTA).

The native elution, consisting of the new protein and its interacting protein partners as well as CBP, can now be analyzed by sodium dodecyl sulfate polyacrylamide gel electrophoresis (SDS-PAGE) or be identified by mass spectrometry.

The invention further relates to an isolated purified protein complex comprising the protein consisting of the amino acid sequence as set forth in SEQ ID NO: 1, or protein derived from the protein as set forth in SEQ ID:1 by substitution, deletion, or insertion of at least one nucleotide provided that said protein retains ubiquitin ligase activity and/or participates to DNA replication, as defined above, and the ORC1 protein or the LRWD1 protein, or both.

In the invention the ORC1 protein consists of the amino acid sequence SEQ ID NO: 57, and the LRWD1 protein consists of the amino acid sequence SEQ ID NO: 58.

The complex as defined above comprises, or consists essentially, or consists of
the protein OBI1 (SEQ ID NO:1) and
the protein ORC1 and/or the protein LRWD1.

This complex may contain nucleic acid molecules.

The invention further relates to a process for identifying compounds destabilizing the complex defined above, said method comprising the following step:
contacting a cell with a compound liable to destabilize the complex as defined above, and
purifying the OBI1 protein as defined above.

According to the above described method, it is possible to screen compounds able to destabilize the above complex, i.e. able to inhibit the interaction between Obi1 and ORC1 or between OBI1 and LRWD1.

The process as defined above is such that, if the purification of OBI1 is not effective, then the tested compound has destabilized the complex, and if the purification of OBI1 is effective, thus the tested compound is not able to destabilize the complex.

One advantageous embodiment of this process is to use fluorescent ORC proteins and OBI1 protein. Such fluorescent proteins are obtained by adding a fluorescent tag. In this embodiment, the fluorescent proteins are able to carry out FRET (Fluorescent rescue energy transfer), such that the emitting wavelength of one of the fluorescent protein corresponds to the absorption wavelength of the other protein.

Therefore, when proteins are close, the complex is detected by exposing cells with a wavelength corresponding to the absorption wavelength of the first fluorescent molecule and by detecting the light with a filter corresponding to the emission wavelength of the second molecule.

If the complex is destabilized, it is therefore impossible to detect the light with a filter corresponding to the emission wavelength of the second molecule, since no FRET is able.

The invention also relates to a method for diagnosing, advantageously in vitro or ex vivo, Meier-Gorlin syndrome, Seckel syndrome and Majewski osteodysplastic primordial dwarfism, comprising a step of identifying the sequence of the Obi1 gene in cells originating from an individual, and a step of comparing said sequence with a reference sequence as set forth in SEQ ID NO: 97.

As demonstrated in Example 12, the inventors have identified in a patient afflicted by Meier-Gorlin syndrome a mutation (a deletion) which leads to a frameshift. The mutation therefore leads to the expression of a truncated protein.

Thus, by identifying the sequence of the Obi1 gene, by sequencing said gene, it is possible to determine if an individual is liable to be afflicted by Meier-Gorlin syndrome. From cells form an individual suspected to be afflicted by Meier-Gorlin syndrome, it is possible to specifically amplify, by PCR, the Obi1 gene, in particular the open reading frame (ORF) of said gene, and thus to sequence this ORF. By comparison with a reference sequence, in particular the sequence as set forth in SEQ ID NO: 97, it is possible to determine if a mutation has occurred, and if said mutation affect the biological function of the resulting protein.

It is also possible to diagnose Meier-Golin syndrome, Seckel syndrome and Majewski osteodysplastic primordial dwarfism, by measuring, possibly in vitro or ex vivo, in cell from an individual suspected to be afflicted by said syndromes, the total activity of the Obi1 protein consisting of the amino acids sequence SEQ ID NO: 1.

If the total Obi1 activity is less that the Obi1 activity of a control cell (originating from healthy individual), preferably at least 1.5× lower than the activity of the control cell, thus the individual is probably afflicted by said syndrome.

The total activity can be measured by determining the Lys6 ubiquitine ligase activity of the proteins co-immuno precipitating with ORC1.

In view of the above description, the skilled person may easily specifically identify such a total Obi1 activity in cells.

The invention advantageously relates to a method for diagnosing Meier-Gorlin syndrome, Seckel syndrome and Majewski osteodysplastic primordial dwarfism, in an individual suspected to have or suspected to further develop said syndrome, said method comprising a step of sequencing the Obi1 cDNA to obtain its sequence, and comparing the obtained sequence with a reference sequence, and in particular with SEQ ID NO: 97.

The skilled person is able to easily determine the above mentioned sequence.

The invention will be better explained in view of the following examples and the following figures.

### BRIEF DESCRIPTION OF THE DRAWINGS

FIGS. 1A-B represent the Tandem affinity Purification (TAP)-TAG approach

FIG. 1 A represents the infection of HeLa S3 wells with retroviruses encoding a tagged version of the protein of interest (bait). Infected cells were expanded after selection of IL2Rα-positive cells. Nuclei were isolated from exponentially growing cells and nuclear extract (Dignam) was prepared.

FIG. 1B represents the Tandem affinity Purification (TAP)-TAG purification process which consists of tandem immunoprecipitation of the baits using the FLAG-HA tag added. Elutions (represented by the arrows) are done by competition with FLAG or HA peptide. Finally, purified complexes were separated on gel and bands were analyzed by mass spectrometry for protein identification.

FIG. 2 represents a silver staining gel showing the proteins contained in the complexes purified according to the TAP-TAG process, by using FLAG-HA tagged ORC1 (A), ORC2 (B) or LRWD1 (C). The proteins that interact with the FLAG-HA tagged proteins are identified by mass spectrometry, and their corresponding name is indicated on the left side of the gel.

FIG. 3 shows a schematic representation of interactions found between the baits (ORC1, ORC2 or LRWD1) and cellular proteins. Several known interactions were rediscovered, including association of the ORC complex with the ubiquitin ligase SCFSkp2, cyclin-dependant kinases (CDK) and the telomere protection complex Shelterin.

FIGS. 4A-B show the Interaction of Obi1 with the ORC complex.

FIG. 4A represents a western blot gel showing proteins contained in TAP-TAG purification samples from HeLa S3 (column 1) or HeLa S3 stably expressing ORC1 FLAG-HA (column 2). Proteins were labeled with specific antibodies; 1.: anti Flag, 2.: anti ORC2, 3.: anti ORC4 and 4.: anti Obi1.

FIG. 4B shows the association between ectopic OBI1 and ORC complex. U20S cells were transiently transfected with tagged Obi1 (column 2) or Obi1 and ORC1 (column 3). Untransfected cells are shown in column 1. The amount of transfected proteins is detected by specific antibodies 1.: anti Myc (detecting Obi1) and 2.: anti FLAG (detecting ORC1). 48 hours post-transfection, cell lysates were subjected to an anti-Flag immunoprecipitation (IP:Flag) (3. and 4.) and the proteins were detected with specific antibodies 3.: anti Myc (detecting Obi1) and 4.: anti FLAG (detecting ORC1)

FIG. 5 shows a schematic representation of the protein and fragments of the invention. The corresponding SEQ ID are also indicated.

FIG. 6 represents the mapping of the Obi1/ORC interaction domains. U20S cells were transfected with the indicated Myc tagged Obi1 protein or fragments, as illustrated in FIG. 5, along with Flag tagged ORC1. Flag-ORC1 immunoprecipitation was performed to monitor specific Obi1 interaction. The co-immunoprecipitation of Obi1 is revealed using an anti Myc antibody (lanes 9 to 16). As control, the amount of immunoprecipitated ORC1 is revealed with an anti-Flag antibody (lanes 17 to 24), and expression of Obi1 is revealed with an anti Myc antibody (lanes 1 to 8). Non-specific bands are marked by an asterisk

FIG. 7 represents the results showing the self-association of Obi1. U20S cells were transfected with the indicated Myc tagged Obi1 protein or fragments, as illustrated in FIG. 5, along with Flag tagged full length Obi1 protein. Flag-Obi1 immunoprecipitation was performed to monitor specific Obi1 dimerization. The co-immunoprecipitation of Myc Obi1 is revealed using an anti Myc antibody (lanes 9 to 16). As control, the amount of immunoprecipitated Flag Obi1 is revealed with an anti-Flag antibody (lanes 17 to 24), and expression of Myc Obi1 is revealed with an anti Myc antibody (lanes 1 to 8). Non-specific bands are marked by an asterisk.

FIGS. 8A-D represents the enhanced licensing induced by Obi1.

FIG. 8A represents flow cytometry histograms showing the DNA content of U20S cells transfected with GFP protein along with 1.: empty vector, 2.: Cdt1 protein, 3.: Cdt1 and Obi1 proteins and 4.: Cdt1 protein and Obi1 devoid of RING domain. Percentages represent the proportion of cells having more than 4n DNA content (re-replicating cells).

FIG. 8B represents the western blots of the transfected cells used in FIG. 8A (columns 1-4). Panel a. represents a blot with an anti Myc antibody (Obi1), panel b. represents a blot with an anti Ha antibody (Cdt1). Panel c. is a control representing a blot with an anti actin antibody.

FIG. 8C blots showing the chromatin loading of the replicative MCM2/MCM7 complex. U20S cells were transfected with 1.: empty vector, 2.: MCM2/MCM5 proteins (Flag tagged proteins), 3.: MCM2/MCM5 (Flag tagged proteins) and Cdt1 protein (Ha tagged protein), and 4.: MCM2/MCM5 (Flag tagged proteins), Cdt1 protein (Ha tagged protein) and Obi1 (Myc tagged protein). The soluble fraction is loaded on gel and revealed with: panel a.: anti-Flag antibody, panel b.: anti-Ha antibody, panel c.: anti Myc antibody. Panel d. is a loading control labeled with an anti actin antibody.

FIG. 8D represents the chromatin fraction of the experiment described in FIG. 8C. panel a. is blotted with an anti-Flag antibody, and panel b., used as control, is blotted with an anti-histones antibody.

FIGS. 9A-E shows that Obi1 behaves like a proto oncogene.

FIG. 9A represents the foci formation assay of NIH 3T3 cells transfected with empty vector.

FIG. 9B represents the foci formation assay of NIH 3T3 cells transfected with Obi1 protein.

FIG. 9C represents the culture in soft agar of NIH 3T3 cells transfected with empty vector.

FIG. 9D represents the culture in soft agar of NIH 3T3 cells transfected with Obi1 protein.

FIG. 9 D represents the protein content (left column: empty vector; right column: transfected with Obi1 protein). Upper panel is a blotting with an anti-Myc antibody revealing the Obi1 protein, and the lower panel is a control of loading.

FIGS. 10A-B shows that ORC3 and ORC5 subunits of the ORC complex is polyubiquitylated via ubiquitin Lys6 linkage in vivo.

FIG. 10A represents a blot of U20S cells transfected with 1: Ha-Ubiquitin alone, or with Ha-Ubiquitin along with the Flag tagged proteins: 2: ORC1, 3: ORC2, 4: ORC3, 5: ORC4, 6: ORC5 and 7: ORC6. After transfection, ORC proteins are immunoprecipitated and the ubiquitin adducts are revealed. The upper blot is a revelation with an anti-Ha antibody and the lower panel is a revelation with an anti-Flag antibody. Bracket represents the Ubiquitin adducts.

FIG. 10B represents a blot of U20S cells transfected with Flag ORC5 along with 1: wild type Ha-Ubiquitin, 2: Ha-Ubiquitin having only the free K6, 2: Ha-Ubiquitin having only the free K48, 2: Ha-Ubiquitin having only the free K63. After transfection, ORC5 protein is immunoprecipitated and the ubiquitin adducts are revealed. The upper blot is a revelation with an anti-Ha antibody and the lower panel is a revelation with an anti-Flag antibody. Bracket represents the Ubiquitin adducts.

FIG. 11 represents the stimulation of ORC polyubiquitylation in vivo by Obi1. U20S cells were transfected with the indicated Myc tagged Obi1 protein or fragments, as illustrated in FIG. 5, along with Flag tagged ORC5. Flag-ORC5 immunoprecipitation was performed to monitor ubiquitination. The ubiquitination is revealed using an anti Ha antibody upper panel). As control, the amount of immunoprecipitated ORC5 is revealed with an anti-Flag antibody (lower panel).

FIG. 12 shows that the activity of Obi1 on ORC polyubiquitylation is conserved. U20S cells were transfected with Flag-ORC5 along with the human (column 2) or the xenopus Obi1 homologs (column 3). 48 hours post-transfection, Flag-ORC5 was immunoprecipitated and probed for the presence of high-molecular weight ubiquitin adducts (upper panel, anti Anti Flag). As control, the expression of human or xenopus Obi1 is evaluated (lower panel, anti Myc).

FIGS. 13A-D shows that Obi1 is the major ORC ubiquitin ligase in vivo.

FIG. 13A represent a gel blotted with an anti-Ha antibody (upper panel; Ubiquitin) or with an anti-Flag (lower panel; ORC3) of an immunipreciptation using an anti-Flag (ORC3) antibody from extracts of U20S cells transfected with Flag ORC3 along with empty vector (column 1), siRNAs against Obi1 (column 3) or a control sequence siRNA (column 2).

FIG. 13B represents a gel blotted with an anti-Myc antibody (upper panel; Obi1) or with an anti-actin (lower panel; control) of total extract of the U20S cells transfected with empty vector (column 1), siRNAs against Obi1 (column 3) or a control sequence siRNA (column 2).

FIG. 13C represent a gel blotted with an anti-Ha antibody (upper panel; Ubiquitin) or with an anti-Flag (lower panel; ORC5) of an immunipreciptation using an anti-Flag (ORC5) antibody from extracts of U20S cells transfected with Flag ORC5 along with empty vector (column 1), siRNAs against Obi1 (column 3) or a control sequence siRNA (column 2).

FIG. 13B represents a gel blotted with an anti-Myc antibody (upper panel; Obi1) or with an anti-actin (lower panel; control) of total extract of the U20S cells transfected with empty vector (column 1), siRNAs against Obi1 (column 3) or a control sequence siRNA (column 2).

FIGS. 14A-D show the importance of Obi1 for efficient cell proliferation and efficient DNA replication

FIG. 14A represents a graph showing cell proliferation of U20S cells transfected with siRNAs against Obi1 (curve with squares) or a control sequence (curve with diamonds). X-axis represents the time in days and Y axis represents the number of cells (×10⁵).

FIG. 14B represents the expression of Obi1 in U20S cells transfected with siRNAs against Obi1 (left column; upper panel) or a control sequence (right column; upper panel). As control, the membrane is blotted with an anti-actin antibody (lower panel).

FIG. 14C represents a graph showing the Bromodeoxyuridine (BrdU) content and the DNA content of cells transfected with non relevant siRNA. G1, S and G2 phases of the cell cycle are indicated.

FIG. 14C represents a graph showing the Bromodeoxyuridine (BrdU) content (Y-axis) and the DNA content (X-axis) of cells transfected with non relevant siRNA. G1, S and G2 phases of the cell cycle are indicated.

FIG. 14C represents a graph showing the Bromodeoxyuridine (BrdU) content (Y-axis) and the DNA content (X-axis) of cells transfected with Obi1 siRNAs. G1, S and G2 phases of the cell cycle are indicated.

FIGS. 15A-D show the subcellular localization of Obi1.

FIG. 15A represents the immunofluorescent result of U20S cells transfected with an empty vector and labelled with an anti-Myc antibody, and revealed with an anti body labelled with FITC.

FIG. 15B represents the immunofluorescent result of U20S cells transfected with a vector expressing Myc-Obi1 and labelled with an anti-Myc antibody, and revealed with an anti body labelled with FITC.

FIG. 15C represents the immunofluorescent result of U20S cells transfected with an empty vector and labelled with Hoescht to label DNA of cells shown in FIG. 15A FIG. 15D represents the immunofluorescent result of U20S cells transfected with a vector expressing Myc-Obi1 and labelled with Hoescht to label DNA of cells shown in FIG. 15B.

FIG. 16 represents a histogram showing the localization of Obi1 in cytoplasm or in the nucleus, scored on at least 100 cells based on the nuclear:/cytoplasm distribution of the signal observed as shown in FIG. 15. White represents a nuclear staining with low cytoplasmic signal (N>C); grey represents cells showing an equal distribution between the nuclear and cytosolic compartment (N=C) and black represents cells having exclusive cytoplasmic signal (N<C).

FIGS. 17A and B show the subcellular localization of Myc-NES-Obi1(1-280).

FIG. 17A represents the immunofluorescent result of U20S cells transfected with a vector allowing the expression of the Myc-NES-Obi1(1-280) protein and labelled with an anti-Myc antibody, and revealed with an anti body labelled with FITC.

FIG. 17B represents the immunofluorescent result of U20S cells transfected with a vector expressing Myc-Obi1 and labelled with Hoescht to label DNA of cells shown in FIG. 17A.

FIG. 18 represents a histogram showing the localization of the Myc-NES-Obi1(1-280) protein in cytoplasm or in the nucleus, scored on at least 100 cells based on the nuclear:/cytoplasm distribution of the signal observed as shown in FIG. 15. White represents a nuclear staining with low cytoplasmic signal (N>C); grey represents cells showing an equal distribution between the nuclear and cytosolic compartment (N=C) and black represents cells having exclusive cytoplasmic signal (N<C).

FIGS. 19A and B show that NES-Obi1(1-280) protein associates with Obi1 protein.

FIG. 19A represents a western blot showing the expression the Myc tagged proteins of U20S cells transfected with Myc-NES-Obi1(1-280) (1.) or Myc-NES-Obi1(1-280) and Flag-Obi1 WT (2.). The blot was labelled with an anti-Myc antibody.

FIG. 19B represents a western blot showing the immunoprecipitation of the Myc tagged proteins co-immunoprecipitated with anti Flag antibody of U20S cells transfected with Myc-NES-Obi1(1-280) (1.) or Myc-NES-Obi1(1-280) and Flag-Obi1 WT (2.). The blot was labeled with an anti-Myc antibody (upper panel). Lower panel is the same blot labelled with an anti Flag antibody.

FIGS. 20A-D shows that the Myc-NES-Obi1(1-280) protein drives Obi1 away from the nucleus.

FIG. 20A represents the immunofluorescent result of U20S cells transfected with an empty vector along with a vector allowing the expression of the Flag-WT Obi1 protein, and labelled with an anti-Flag antibody, and revealed with an anti body labelled with FITC.

FIG. 20B represents the immunofluorescent result of U20S cells transfected with a vector expressing the Myc-NES-Obi1(1-280) protein along with a vector allowing the expression of the Flag-WT Obi1 protein, and labelled with an anti-Myc antibody, and revealed with an anti body labelled with FITC.

FIG. 20C represents the immunofluorescent result of U20S cells transfected with an empty vector along with a vector allowing the expression of the Flag-WT Obi1 protein, and labelled with Hoescht to label DNA of cells shown in FIG. 20A

FIG. 20D represents the immunofluorescent result of U20S cells transfected with a vector expressing the Myc-NES-Obi1(1-280) protein along with a vector allowing the expression of the Flag-WT Obi1 protein, and labelled with Hoescht to label DNA of cells shown in FIG. 20B.

FIG. 21 represents histograms showing the localization of the Myc-NES-Obi1(1-280) protein in cytoplasm or in the nucleus, scored on at least 100 cells based on the nuclear:/cytoplasm distribution of the signal observed as shown in FIGS. 20A and C (A) or FIGS. 20B and D (B). White represents a nuclear staining with low cytoplasmic signal (N>C); grey represents cells showing an equal distribution between the nuclear and cytosolic compartment (N=C) and black represents cells having exclusive cytoplasmic signal (N<C).

FIGS. 22A-C shows that the expression of NES-Obi1(1-280) induces a selective loss of transfected cells of the transfected vectors.

FIG. 22A represents a graph measuring the intensity of GFP fluorescence (X-axis) of U20S cells transfected with empty vectors.

FIG. 22B represents a graph measuring the intensity of GFP fluorescence (X-axis) of U20S cells transfected with an empty vector and a vector allowing the expression of the fluorescent EGFP protein.

FIG. 22C represents a graph measuring the intensity of GFP fluorescence (X-axis) of U20S cells transfected with a vector expressing the Myc-tagged NES-Obi1(1-280) protein and a vector allowing the expression of the fluorescent EGFP protein.

FIGS. 23A-B show the DNA and protein sequence of Obi1 in a patient afflicted by Meier-Gorlin syndrome.

FIG. 23A represents the alignment of wild-type and mutated Obi1 coding sequences (SEQ ID NO: 101 and 104). The corresponding amino acids are also shown (SEQ ID NO: 102 and 103). The mutation is a two nucleotides deletion (AA, underlined) resulting in a frameshift and a premature STOP codon (* in SEQ ID NO; 104).

FIG. 25B represents the position of the mutation in a schematic representation of the Obi1 protein. The mutation, leading to a truncation, maps within the C-terminal domain of Obi1 (indicated by an arrow).

FIGS. 24A-B show that the mutated Obi1 found in the patient is impaired for ORC association.

FIG. 24A represents a western blot showing the expression of WT Obi (1) and mutated Obi1 (2) proteins, when co-expressed (+) or not (−) with Flag-ORC1 protein (#1). Proteins were revealed by using anti-Myc antibody. Numbers (100, 80, 60) represent the molecular mass in kDa.

FIG. 24B represents a western blot of a co-immunoprecipitation of Obi1 proteins (WT:A; mutated: B—panel 1.) by using an anti-Flag antibody that immunoprecipitates Flag-ORC1 protein.). Proteins were revealed by using anti-Myc antibody. Numbers (100, 80, 60) represent the molecular mass in kDa. Panel 2. shows the immunoprecipitated ORC1 proteins. Proteins were revealed by using anti-Flag antibody

### EXAMPLES

### Example 1

### Materials and Methods

### Cell Culture, Transfections and Infections

U20S, HeLa S3, NIH 3T3 and Platinum-E (Cell Biolabs) cells were grown in DMEM (Invitrogen) supplemented with 10% Foetal Bovine Serum (FBS), 2 mM glutamine and antibiotics. Cells were transfected using the Lipofectamine reagent (Invitrogen) as specified by the manufacturer. For TAP-TAG experiments, HeLa S3 cells stably expressing baits were generated by retroviral transduction. For this, viral particles were generated using Platinum-E ecotropic packaging cell line transiently transfected with the retroviral constructs. In parallel, HeLa S3 cells were transiently transfected with mouse Cationic Aminoacid Transporter 1 to render them susceptible to infection by ecotropic retroviruses. 48 hours after transfection, S3 cells were incubated with viral supernatant in presence of polybrene (10 um/mL, Sigma Aldrich) for 24 hours. 48 hours later, transduced cells express human IL2 receptor alpha chain which allows selection using mouse anti-IL2Ra antibody (Upstate Biotechnology) coated magnetic beads as specified by the manufacturer (Dynabeads M-450 Epoxy, Invitrogen). Knockdown experiments were done using a pool of 4 siRNAs (Smartpool, Dharmacon) targeting human Obi1 transfected using Interferin reagent (PolyPlus Transfection) as specified by the manufacturer. 10 nM siRNA was added to the cells.

### Antibodies

Anti-Flag M2 [# F1804; 1/1000], anti-Actin [# A5441; 1/5000] and anti-PCNA [# WH0005111M2; 1/2500] monoclonal antibodies were from Sigma. The anti-Myc 9B11 monoclonal antibody [#2276S; 1/1000] was from Cell Signaling Technologies. The polyclonal antibodies against HA [# sc-805; 1/100], Obi1/RNF219 [# sc-84039; 1/100] and ORC4 [# sc-20634; 1/100] were from Santa-Cruz BioTechnologies. The anti-ORC2 antibody [ 1/1000] was described previously (Cayrou et al. (2011) Genome Res 21:1438-49.)

### Plasmid Constructs

For TAP-TAG experiments, human ORC1, ORC2 and LRWD1 were amplified by PCR using cloned cDNA as template with the following primers:

Orc1CnotS:
(SEQ ID NO: 64)
CTGGCGGCCGCACCATGGCACACTACCCCACAAGG
Orc1CnotAS:
(SEQ ID NO: 65)
CTGGCGGCCGCCTCGTCTTTCAGCGCATAC
Orc2CnotS:
(SEQ ID NO: 66)
CTGGCGGCCGCACCATGAGTAAACCAGAATTAAAGGAAGA
Orc2CnotAS:
(SEQ ID NO: 67)
CTGGCGGCCGCAGCCTCCTCTTCTTCCTTTTC
LrwdCnotS:
(SEQ ID NO: 68)
CTGGCGGCCGCACCATGGGCCCCCTCTCGG
LrwdCnotAS:
(SEQ ID NO: 69)
CTGGCGGCCGCCATCCTCCCCCAGATGGC.

These primers introduced a Not I restriction enzyme site at both end of the cDNA. Using Pfu polymerase (Promega) as specified by the manufacturer: The following PCR conditions were:

### 1) 2 min at 95° C.

### 2) 30 sec at 95° C.

### 3) 30 sec at 58° C.

4) 2 min/kb extension time at 73° C.
5) repeat step 2-4 25 times

### 6) 5 min at 74° C.

### 7) End, 4° C.

PCR products were purified (Qiaquick, Qiagen) and digested with NotI. The DNA fragments were subcloned at the Not I site of pOZ-FH-C to produce a protein having an inframe C-Terminal Flag-HA tag. Restriction digestion and sequencing analysis allowed us to screen for the proper construction. For co-immunoprecipitation and ubiquitination studies, a vector expressing an N-Terminal Flag tag was constructed. The pCS2 vector was digested with BamHI/EcoRI fragment, which removes the Myc6 tag and ligated with the following annealed oligos:

FlagBamNcoEcoS:
(SEQ ID NO: 70)
GATCCGCCAACATGGACTACAAGGACGACGATGACAAGTCCATGGCT
GGG
FlagBamNcoEcoAS:
(SEQ ID NO: 71)
AATTCCCAGCCATGGACTTGTCATCGTCGTCCTTGTAGTCCATGTTG
GCG

This yields pCS2-Flag. Then, ORC1-6 were amplified by PCR using the cloned cDNA as template with the following template:

ORC1_XbaS:
(SEQ ID NO: 72)
CTGTCTAGAGCCATGGCACACTACCCCACAAGGC
ORC1_XbaAS:
(SEQ ID NO: 73)
CTGTCTAGATTACTCGTCTTTCAGCGCATAC
ORC2_NcoS:
(SEQ ID NO: 74)
CTGCCATGGGAAGTAAACCAGAATTAAAGGAAGAC
ORC2_XhoAS:
(SEQ ID NO: 75)
CTGCTCGAGTCAAGCCTCCTCTTCTTCCTT
ORC3_NcoS:
(SEQ ID NO: 76)
CTGCCATGGCTACGTCCTCGAT
ORC3_XbaAS:
(SEQ ID NO: 77)
CTGTCTAGATTAGCAGCCTCCCCATGTTAG
ORC4_XhoS:
(SEQ ID NO: 78)
CTGCTCGAGAGCAGTCGTAAATCAAAGAG
ORC4_XbaAS:
(SEQ ID NO: 79)
CTGTCTAGATTATAACCAGCTTAGTGAGGATG
ORC5_EcoS:
(SEQ ID NO: 80)
CTGGAATTCTCCCCACTTGGAAAACGTGG
ORC5_XhoAS:
(SEQ ID NO: 81)
CTGCTCGAGTCACAAGAAATCATACAAGTATTT
ORC6_XhoS:
(SEQ ID NO: 82)
CTGCTCGAGGGGTCGGAGCTGATCG
ORC6_XbaAS:
(SEQ ID NO: 83)
CTGTCTAGATTACTCTGCTGTAGCCTTTTGA.

These primers introduced the indicated sites at both end of the cDNA. Using Pfu polymerase (Promega) as specified by the manufacturer: The following PCR conditions were:

### 1) 2 min at 95° C.

### 2) 30 sec at 95° C.

### 3) 30 sec at 58° C.

4) 2 min/kb extension time et 73° C.
5) repeat step 2-4 25 times

### 6) 5 min at 74° C.

### 7) End, 4° C.

PCR products were purified (Qiaquick, Qiagen) and digested with the appropriate restriction enzymes. The DNA fragments were subcloned at the relevant site in pCS2-FLAG vector. Restriction digestion and sequencing analysis allowed us to screen for the proper construction. To construct human Myc6-tagged Obi1 WT expression vectors, we amplified by PCR using the cloned cDNA as template with the following template:

Rn_1_NcoS:
(SEQ ID NO: 84)
CTGCCATGGCTCAGACCG
Rn_Stop_XbaAS:
(SEQ ID NO: 85)
CTGTCTAGATTAACTTTTAGTTGCTTTTGATGGG

For Obi1 ΔRING, the following primer were used:

Rn_54_NcoS:
(SEQ ID NO: 86)
CTGCCATGGCTTGCAGAGTCCCCATCA
Rn_Stop_XbaAS:
(SEQ ID NO: 85)
CTGTCTAGATTAACTTTTAGTTGCTTTTGATGGG

For Obi1 ΔCTD/OBD, the following primers were used:

Rn_1_NcoS:
(SEQ ID NO: 84)
CTGCCATGGCTCAGACCG
Rn_280XbaAS:
(SEQ ID NO: 87)
CTGTCTAGATTATTTGCCATCTGCAGAAAGTG

For Obi1 CC, the following primers were used:

Rn_54_NcoS:
(SEQ ID NO: 86)
CTGCCATGGCTTGCAGAGTCCCCATCA
Rn_280XbaAS:
(SEQ ID NO: 87)
CTGTCTAGATTATTTGCCATCTGCAGAAAGTG

For Obi1 CTD/OBD, the following primers were used:

Rn_280NcoS:
(SEQ ID NO: 88)
CTGCCATGGGGAGCAAAGGCAGTGAG
Rn_Stop_XbaAS:
(SEQ ID NO: 85)
CTGTCTAGATTAACTTTTAGTTGCTTTTGATGGG

These primers introduced the indicated sites at both end of the cDNA. Using Pfu polymerase (Promega) as specified by the manufacturer: The following PCR conditions were:

### 1) 2 min at 95° C.

### 2) 30 sec at 95° C.

### 3) 30 sec at 58° C.

4) 2 min/kb extension time et 73° C.
5) repeat step 2-4 25 times

### 6) 5 min at 74° C.

### 7) End, 4° C.

PCR products were purified (Qiaquick, Qiagen) and digested with NcoI/XbaI. The DNA fragments were subcloned at the relevant site in pCS2.

To construct ObiΔCC, the RING domain of Obi1 was amplified with the following primer:

Rn_1_NcoS:
(SEQ ID NO: 84)
CTGCCATGGCTCAGACCG
Rn_73NcoBAS:
(SEQ ID NO: 89)
CTGCCATGGATCCTCCTATAATTTCTTTGCAA

and cloned at the NcoI of pCS2-Obi1 CTD/OBD. Restriction digestion and sequencing analysis allowed us to screen for the proper construction. To construct Xenopus Myc6-tagged Obi1 WT expression vectors, we amplified by PCR using the cloned cDNA as template with the following template:

RnXenoEcoS:
(SEQ ID NO: 90)
CTGGAATTCTATGGCGCAGACTGTTCAGAA
RnXenoXhoAS:
(SEQ ID NO: 91)
CTGCTCGAGTCATGGCTTAAAGGACTTTGGAG

PCR products were purified (Qiaquick, Qiagen) and digested with EcoRI/XhoI. The DNA fragments were subcloned at the relevant site in pCS2. HA-tagged ubiquitin WT and K mutants are described (Sobhian et al. (2007) Science 316:1198-202.). For re-replication experiments, human WT Cdt1 with an C-Terminal HA-tag was generated.

First, pcDNA3 (Invitrogen) was digested with EcoRI/XbaI and the following annealed oligos were cloned:

HAEcoXbaS:
(SEQ ID NO: 92)
AATTCTATGTATGATGTTCCTGATTATGCTAGCCTCTAAT
HAEcoXbaAS:
(SEQ ID NO: 93)
CTAGATTAGAGGCTAGCATAATCAGGAACATCATACATAG

To yield pcDNA3-HACT. Then, human WT Cdt1 was amplified by PCR using cloned cDNA as template with the following primers:

hCdt1-Cter-HdS:
(SEQ ID NO: 94)
CTGAAGCTTACCATGGAGCAGCGCCGC
hCdt1-Cter-EcoAS:
(SEQ ID NO: 95)
CTGGAATTCAGCCCCTCCTCAGCAC.

PCR products were purified (Qiaquick, Qiagen) and digested with HindIII/EcoRI. The DNA fragments were subcloned at the relevant site in pcDNA3-HACT. pEGFP-C2 (Clontech) was used an EGFP expression vector. In chromatin fractioning experiments, Flag-tagged human MCM2 and MCM5 have been described (Tardat et al (2010) Nat Cell Biol 12:1086-93.)

### Re-Replication Assay

U20S cells were co-transfected with the tested constructs and an expression plasmid coding for EGFP. 60-72 h post-transfection, cells were trypsinized and fixed in 1% paraformaldehyde in PBS at RT for 15 min. Washed cells were fixed again in 70% ethanol in PBS and stored at −20° C. overnight. Cells were spun at 2000 rpm for 10 min, washed in PBS and treated with 50 μg/mL RNAse A. DNA was stained with 25 μg/mL propidium iodide. Cells were analysed with a FACSCalibur flow cytometer using the CellQuestPro software.

### Protein Biochemistry

For cell lysis, immunoprecipitation and western blotting were performed following standard methods. Briefly, cells were lysed in TK300 lysis buffer (20 mM Tris-HCl pH 7.9; 300 mM KCl, 5 mM MgCl2, 10% glycerol; 0.5% NP-40 plus protease inhibitors cocktail) on ice for 30 min. The protein concentration of the clarified lysates was estimated using the BCA method (Pierce). For immunoprecipitation experiments, equivalent amounts of protein samples were incubated at 4° C. with anti-Flag coupled agarose beads (Sigma Aldrich) for 4 h. After extensive washing with TK300 lysis buffer, bound proteins were eluted in boiling Laemmli buffer. Samples were runs on pre-cast gradient gel (NuPage Novex, Invitrogen and Mini-PROTEAN TGX, Biorad) and transferred to nitrocellulose membrane. Western blot were revealed using ECL prime (Amersham).

### In Vivo Ubiquitination Assay

U20S cells were co-transfected with Flag-tagged ORC subunits along with HA-ubiquitin (WT or K only mutants). In some cases, Obi1 (WT or mutants) was co-transfected. In other cases, endogenous Obi1 expression was silenced by treated the cells with Obi1 siRNAs 24 hours before plasmid transfection. 48 hours after plasmid transfection, cells were lysed in Triton X-100 lysis buffer (50 mM Tris-HCl pH=7.4; 100 mM NaCl; 50 mM NaF; 5 mM EDTA; 40 mM β-glycero-phosphate; 1% Triton X-100 plus protease inhibitors cocktail) supplemented with 10 mM N-ethyl maleimide (NEM, Sigma) to inhibits deubiquitinating enzymes. Cell lysates (750 μg protein) were then incubated for 2 h at 4° C. with anti-Flag coupled agarose beads (Sigma Aldrich). The bound proteins were washed 5 times in lysis buffer and resolved on gradient gels. Ubiquitin-containing conjugates were detected by immunoblotting with anti-HA antibody.

### Chromatin Isolation

Chromatin and soluble fractions were obtained using the CSK procedure. Briefly, cells were lysed in CSK buffer (10 mM PIPES pH=6.8; 100 mM NaCl; 300 mM sucrose; 1 mM MgCl2; 0.5 mM DTT; 0.2% Triton X-100; 1 mM ATP plus protease inhibitor cocktail) on ice for 10 min. Insoluble material was pelleted (3000 rpm, 4° C., 3 min). Supernatants (soluble fraction) were kept, while pellets were extracted a second time in CSK buffer. The final pellets (chromatin fraction) were solubilized in Laemmli buffer.

### Foci Formation Assay

NIH 3T3 cells stably expressing human Obi1 or the empty vector were plated in duplicate in 60 mm plates at equivalent density. Confluent cells were cultured for 2-3 weeks and the culture medium was changed every 2-3 days. Foci were stained with crystal violet dye.

### Soft Agar Growth Assay

105 cells of NIH 3T3 cells stably expressing human Obi1 or the empty vector were mixed with medium supplemented with 0.4% noble agarose (sigma) and placed on top of the 1% agarose layer. 1 mL medium was added to the solidified layer and changed every 2-3 days. After 3-4 weeks, colonies were counted in 10 randomly selected fields using a phase contrast microscope.

### Preparation of Nuclear Extracts (Dignam Method)

Hela S3 cells (40*150 mm confluent dishes) were trypsinized to obtain a homogenous cell suspension. After a wash in PBS, cells were lyzed in a hypotonic buffer (HB buffer: 10 mM Tris pH 7.9; 1.5 mM MgCl2; 10 mM KCl; 1 mM DTT; 10 mM PMSF). Briefly, cells were washed once in ice cold HB buffer (10 times the cell pellet volume (CPV). The pelleted cell pellet was then resuspended in 2 times CPV of HB buffer. Cells were incubated on ice for 10-15 min. Swollen cells are transferred to a dounce homogenizer and cells were lyzed by 15 stroke using the tight pistol. The nuclei are pelleted by centrifugation. The supernatant is discarded and the nuclei are resuspended in 0.5 nuclear pellet volume (NPV) of Low Salt (LS) buffer (20 mM Tris pH 7.9; 1.5 mM MgCl2; 0.2 mM EDTA; 20 mM KCl; 1 mM DTT; 10 mM PMSF). Whilst gently vortexing the tube, add 0.5×NPV of High Salt (HS) buffer (20 mM Tris pH 7.9; 1.5 mM MgCl2; 0.2 mM EDTA; 1200 mM KCl; 1 mM DTT; 10 mM PMSF) drop by drop. After a 30 min incubation with agitation in the cold room, the Dignam nuclear extracts are clarified by centrifugation and freeze in liquid nitrogen. The extracts were kept at −80 C

### TAP-TAG Procedure

All procedures were done at 4 C and on ice. Thaw Dignam extract on ice for 30-45 min. Spin (13 000 rpm, 20 min) to clarify the extract. A typical experiment involves ˜10 mL of extract. We first performed the Flag IP. We use 1% volume/volume of anti-Flag coupled agarose beads (Sigma, ˜100 μL). Beads were washed to diminish background binding: ¼ volume beads of 0.1 M Glycine pH=2.2 was added to the beads and incubate for 3 min at room temperature. ½ volume beads of Tris 1M pH=7.9 was added. The beads were washed twice with 1 mL of HA-IP Buffer (150 mM KCl; 20 mM Tris pH 7.5 0.05% NP40; 0.1% Tween; 10% Glycerol; 5 mM MgCl₂; 1 mM DTT and 10 mM PMSF). After the last centrifugation, beads were resuspended in HA-IP Buffer (1 vol of Beads). Extracts were incubated with Flag beads for 4 h with agitation at 4 C. Beads were pelleted (3 min at 3000 rpm 4° C.). Beads were washed 5 times with HA IP Buffer: Beads are pelleted (3K 3 min 4° C.) and the supernatant discarded. Complexes were eluted from the beads by incubating in 3 beads volume of HA IP Buffer supplemented with Flag peptide (200 μg/mL, Sigma) for 1 hour at 4 C with agitation. The supernatant was kept on ice and a second elution was conducted. The eluates were pooled and subjected to an anti-HA immunoprecipitation. Before, anti-HA coupled agarose beads (SantaCruz Biotechnology) were washed with glycine buffer as described above. 10 μL of beads was used per IP. Flag eluates were incubated with anti-HA beads for 2 hours at 4 C with agitation. Beads were pelleted (3 min at 3000 rpm 4° C.). Beads were washed 5 times with HA IP Buffer: Beads are pelleted (3K 3 min 4° C.) and the supernatant discarded. Complexes were eluted from the beads by incubating in 3 beads volume of HA IP Buffer supplemented with HA peptide (400 μg/mL, Roche) for 1 hour at 4 C with agitation. The supernatant was keep on ice and a second elution was conducted. HA eluates were clarified on a filter column (Pierce Spin Cups Cellulose Acetate Filter ThermoFisher): columns were rinsed with 400 μl of HA IP buffer and spun for 1 min at 7000 rpm at 4° C. The supernatant was discarded and eluates were apply to the column. After spinning, the flow-through was collected and denatured in lammeli buffer. 25% of the samples were run in precast NuPage gradient gel and stained by silver nitrate (SilverQuest, Invitrogen) as specified by the manufacturer. 75% of samples were run on precast gel and stained with colloidal blue staining kit (Invitrogen) as specified by the manufacturer.

### Mass Spectrometry Analysis

Bands on colloidal blue stained gels were excised, reduced with DTT and alkylated with lodoacetamine (IAA) using standard procedure. Bands were then subjected to trypsin (Promega) digestion. Extracted peptides were collected and analysed using a nanoESI LTQ_XL Orbitrap mass spectrometer (Thermo Fisher Scientific) coupled to an Ultimate 3000 HPLC (Dionex). A gradient of 0-40% B for 30 min and 80% B for 15 min (A=0.1% formic acid, 2% acetonitrile in water; B=0.1% formic acid in acetonitrile) at 300 nl/min was used to elute peptides from the capillary (0.075 mm×150 mm) reverse-phase column (Pepmap®, Dionex). Nano-ESI was performed with a spray voltage of 2.4 kV, heated capillary temperature of 200° C., and tube lens voltage of 140 V. A cycle of one full-scan mass spectrum (400-1600 m/z) at a resolution of 30000, followed by five data-dependent MS/MS spectra was repeated continuously throughout the nanoLC separation. All MS/MS spectra were recorded using normalized collision energy and an isolation window of 2 m/z. Data were acquired using the Xcalibur software (v 2.0.7, Thermo Fisher Scientific). Data analysis was performed using MaxQuant 1.3.0.5 and standard parameters against the CPS_HUMAN database (November 2012). FDR was set at 1%.

### Example 2

### Purification of the OBI1 Protein

Identification of the interaction partners of a particular protein became a valuable way to gain insight into the physiological role of the protein.

The most widely used system to identify intermolecular interactive partners of a particular protein of interest is the yeast two-hybrid system. However, given that this system is designed to detect mostly binary interactions, it is not suitable for investigating the intermolecular interaction partners of a protein if the latter is a component of a multimeric protein complex.

For identification of intermolecular interaction partners, purification of the native protein complexes from the cells would be the best method. This would provide information on how the endogenous protein interacts with intermolecular interaction partners.

However, in general, purification of native complexes is technically difficult and time-consuming, especially when the protein of interest is scarce. These limitations can be addressed with epitope tagging. It has been shown that stably expressed exogenous proteins with epitope tags are integrated into the complexes when the complex is synthesized de novo. Such a protein complex can be purified rapidly by immunoprecipitation with antibodies against epitopes.

The inventors took advantage of the epitope-tagging technique to develop a standardized approach to purifying multimeric protein complexes from mammalian cells. We put two different epitope tags tandemly (i.e., FLAG and HA) on the C terminus of the proteins of interest. For rapid generation of cell lines that express the epitope-tagged proteins of interest, the inventors used retroviral transduction. The cells are sorted magnetically after transduction. Once stably transduced cell lines are established, the protein complex can be purified from cells in less than 24 h.

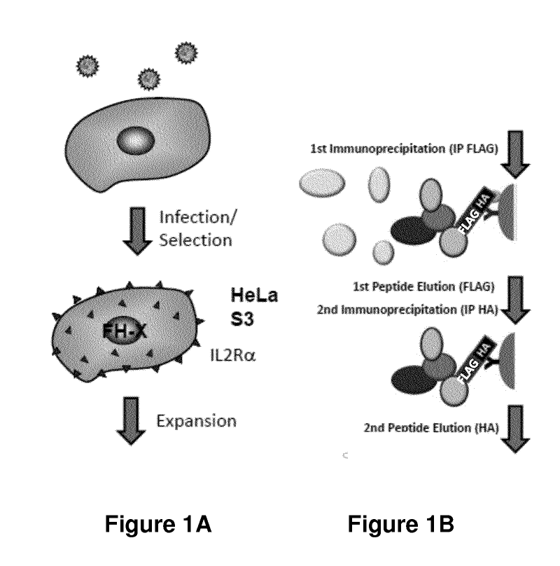

The above steps are represented in FIGS. 1A-B.

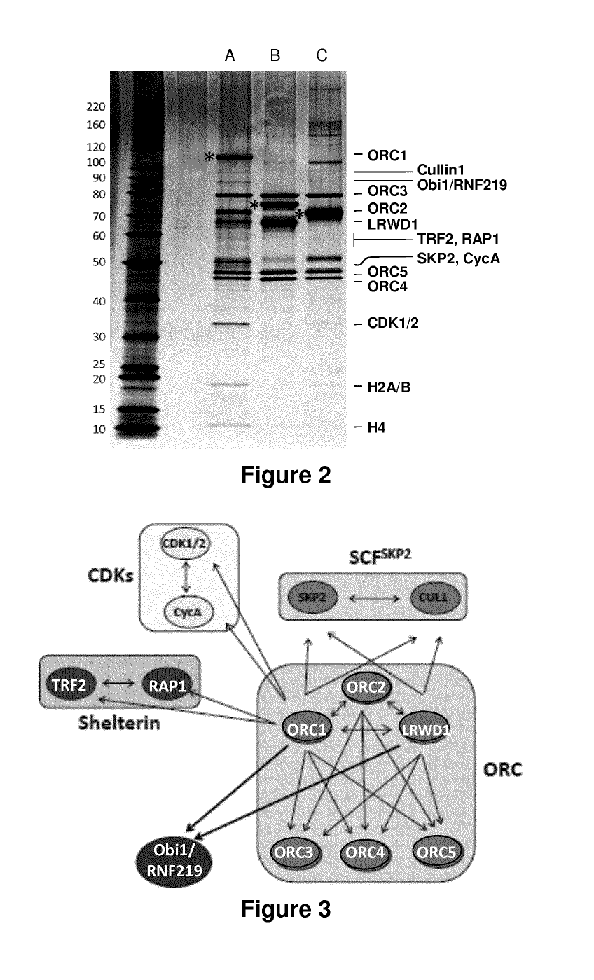

Results of TAP-TAG assays are reproduced on FIG. 2. Nuclear extract from HeLa S3 cells stably expression the indicated proteins were subjected to the Tandem-Affinity Purification (TAP)-TAG approach. Purified complexes were visualized by Silver staining and Mass Spectrometry for protein identification (shown on the left side).

These data demonstrates that Obi1/RFN219 interacts specifically with ORC1 and LRWD1, but is undetectable for ORC2.

FIG. 3 summarize the ORC complex interactions.

### Example 3

### OBI1 Protein Interacts with ORC1

In order to validate the TAP-TAG interaction, the inventors have tested the Obi1-ORC1 interaction in vitro by co-immunoprecipitation.

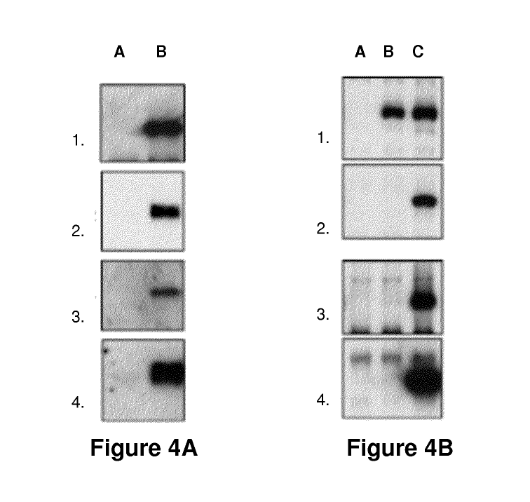

Results are shown in FIGS. 4A-B.

As shown, immunoprecipitation of ORC1 co-immunoprecipitate Obi1 protein, confirming the interaction observed in TAP-TAG assay.

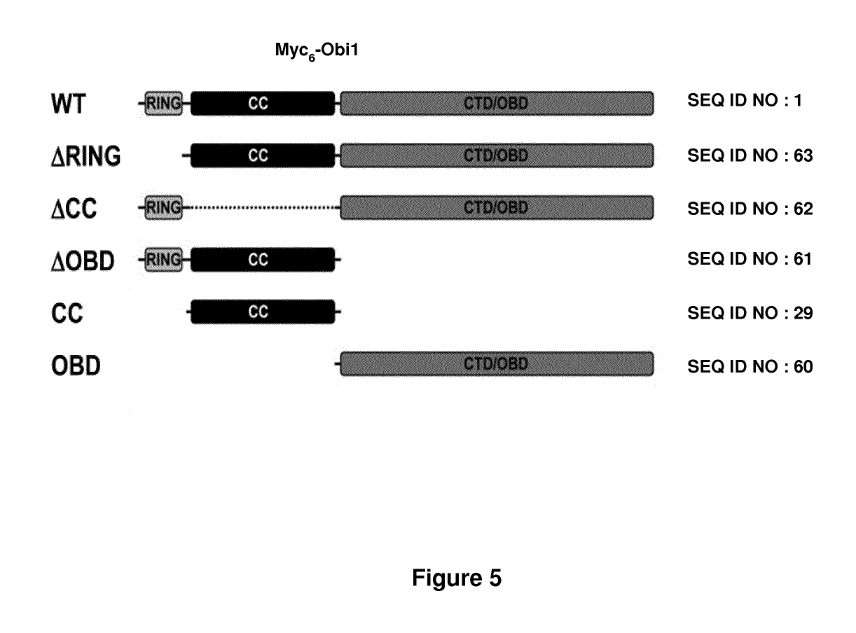

Further, to determine Obi1 domains involved in ORC1/Obi1 interaction, the inventors have constructed Obi1 deletion fragments of Obi1 and evaluated their ability to interact with ORC1. Constructions are shown in FIG. 5.

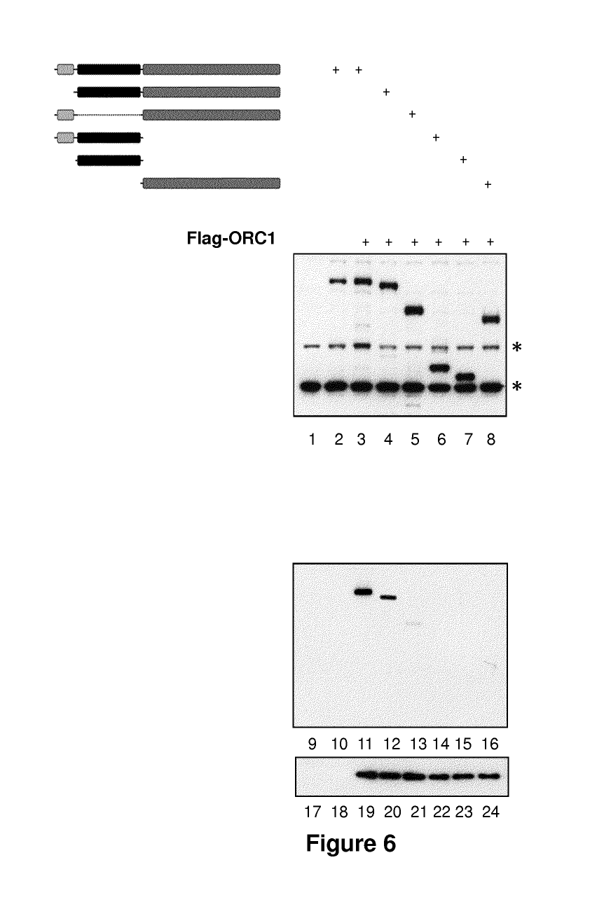

FIG. 6 shows the results. In vitro, Obi1 requires its C-terminal Domain (CTD) and Coiled-coil (CC) region, but not its catalytic RING domain for ORC association.

### Example 4

### OBI1 Self-Associates

To further identify biochemical properties of Obi1, the inventors have evaluated its ability to self-associate.

The deletion mutants described in FIG. 5 were co-immunoprecipitated with full length Obi1 protein.

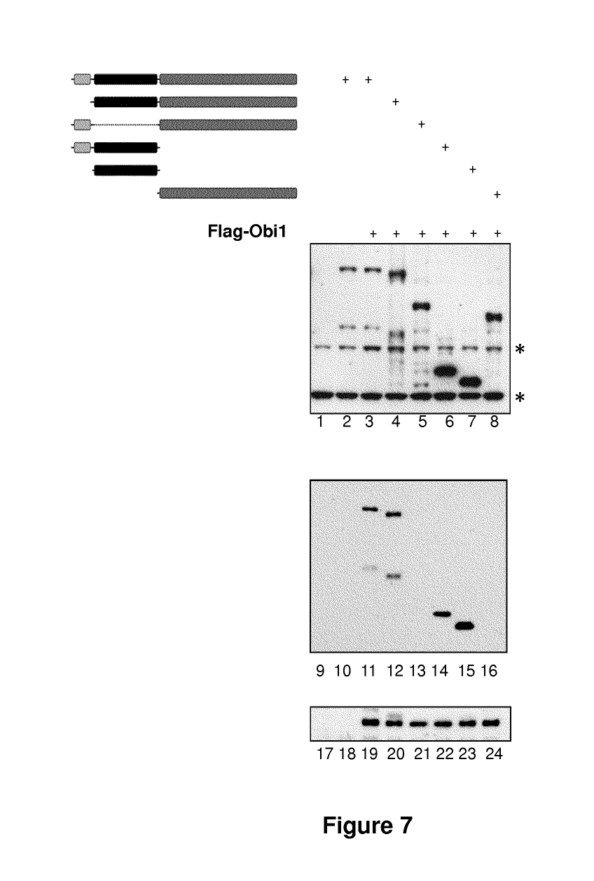

Results are shown in FIG. 7.

It is to be noticed that all constructs having the Coiled-coil region can dimerize with WT Obi1.

### Example 5

### OBI1 Enhances Licensing Activity

Since ORC1 participate to DNA replication, the inventors have evaluated Obi1 function during DNA replication.

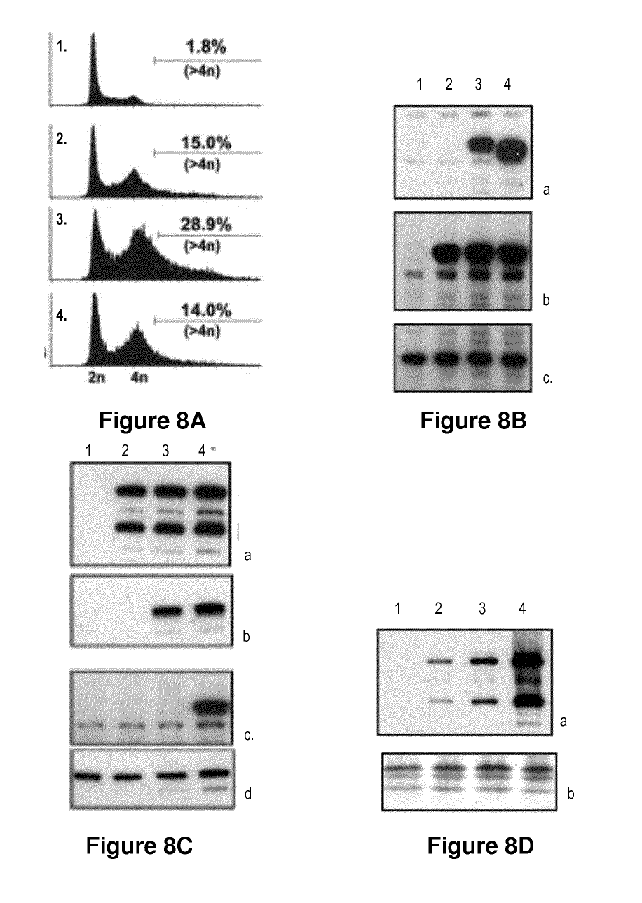

In FIG. 8A, it is demonstrated that Obi1 stimulates re-replication induced by Cdt1 overexpression, since the fraction of cells having DNA content >4n is enhanced when Obi1 is over expressed, said effect requiring the presence of the RING domain of Obi1. In FIG. 8D, it is shown that Obi1 stimulates the chromatin loading of MCM2-7 complex, as shown by the MCM2/5 amount in chromatin in cells transfected with both Obi1 and Cdt1.

### Example 6

### OBI1 Behaves Like a Proto-Oncogene

Since Obi1 participate to DNA replication by inducing re-replication of DNA, the inventors have investigated the oncogenic potential of Obi1.

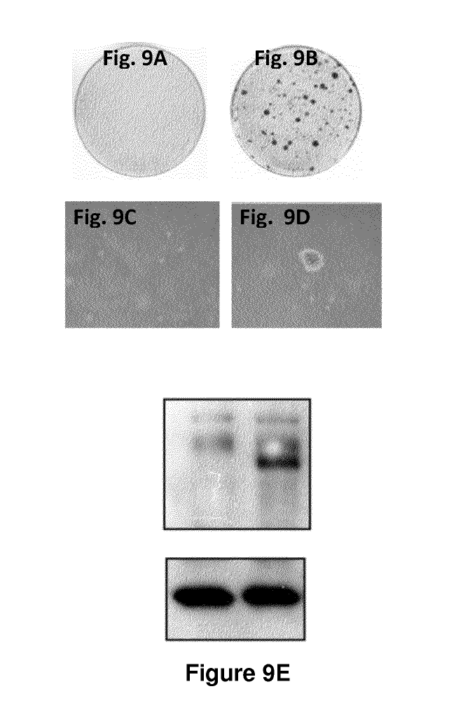

FIG. 9 show that NIH 3T3 cells stably overexpressing Ob1 protein are able to form foci on plates and are able to form colonies when cultured in soft agar whereas the control non-transformed cells are not. Both these assays respectively demonstrate that Obi1 deregulated expression allows the loss of contact inhibition and allows the anchorage independent growth, which are hallmarks of cancer cells.

These data demonstrate that Obi1 behaves like a proto-oncogene.

### Example 7

### ORC Complex is Polyubiquitylated Via Ubiquitin Lys6 Linkage In Vivo

Since Obi1 protein contains a RING domain, the inventors have evaluated its participation in protein ubiquitination process.

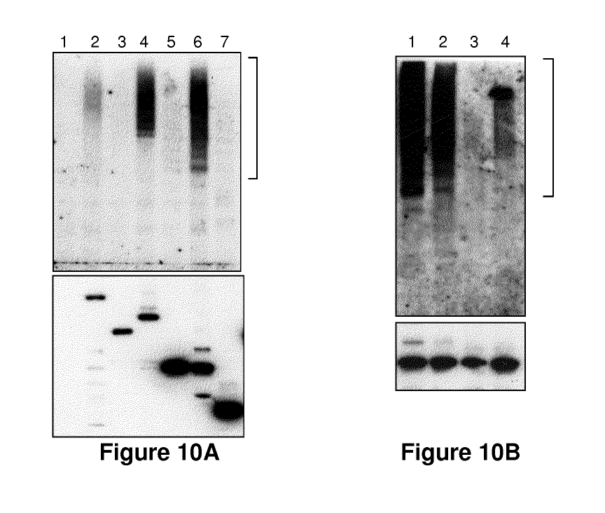

The inventors have first evaluated the ubiquitination of ORCs protein in vitro in presence of Obi1 protein. FIG. 10A shows that OC1, ORC3 and ORC5 are ubiquitinated in presence of Obi1, and in particular that ORC3 and ORC5 show strong polyubiquitylation.

In a second time, the inventors have demonstrated that ubiquitination is essentially carried out via Ubiquitin Lys6. Indeed, as shown in FIG. 10B, cells transfected with K6-only Ubiquitin mutant are note able to ubiquitinate ORC5, when transfected with Obi1.

These data demonstrate that Obi1 is a K6 polyubiquitin ligase.

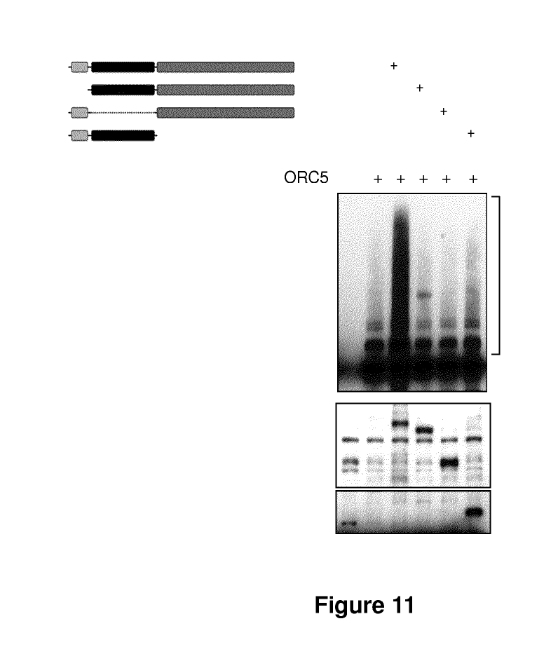

In vitro experiments, shown in FIG. 11, demonstrate that only the full length Obi1 protein is able to polyubiquitinate ORC5.

The inventors have tested if the poly-ubiquitin ligase activity of human Obi1 protein is conserved.

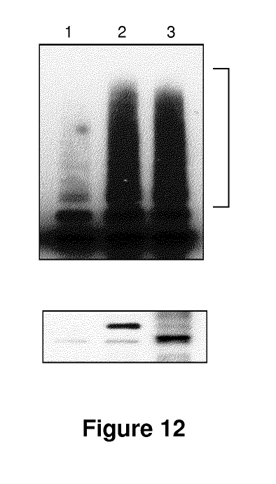

As shown in FIG. 12, both human and xenopus Obi1, sharing 35% identity of their amino acid sequence, are able to ubiquitinate ORC5.

These results demonstrate that the activity of Obi1 on ORC polyubiquitylation is conserved toward species.

### Example 8

### Obit is the Major ORC Ubiquitin Ligase In Vivo

To determine the importance of the Obi1 in poly-ubiquitination of ORC, the inventors have invalidated Obi1 gene by RNA interference.

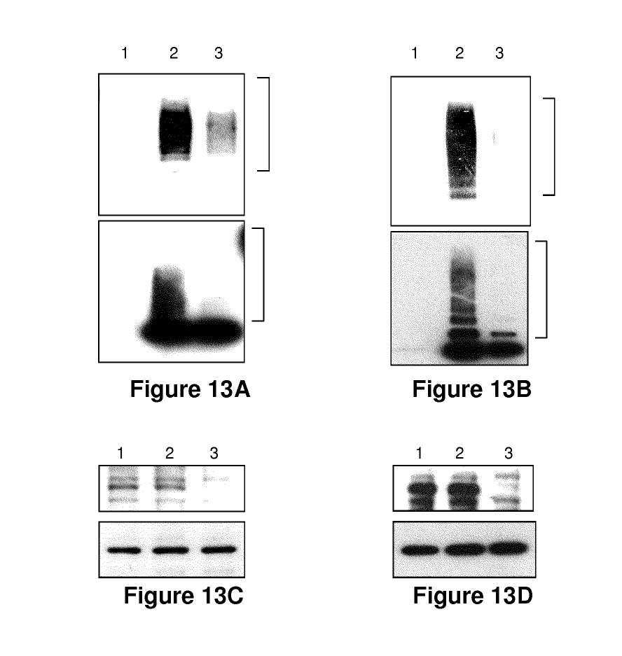

FIG. 13 shows that cells that does not express Obi1 are not able to poly-ubiquitinate either ORC3 or ORC5.

Obi1 is thus the major ORC ubiquitin ligase in vivo.

### Example 9

### Obi1 is Required for Efficient Cell Proliferation and Efficient DNA Replication

To further evaluate the function of Obi1, the inventors have evaluated the proliferation of cells in which Obi1 expression has been inhibited by siRNAs.

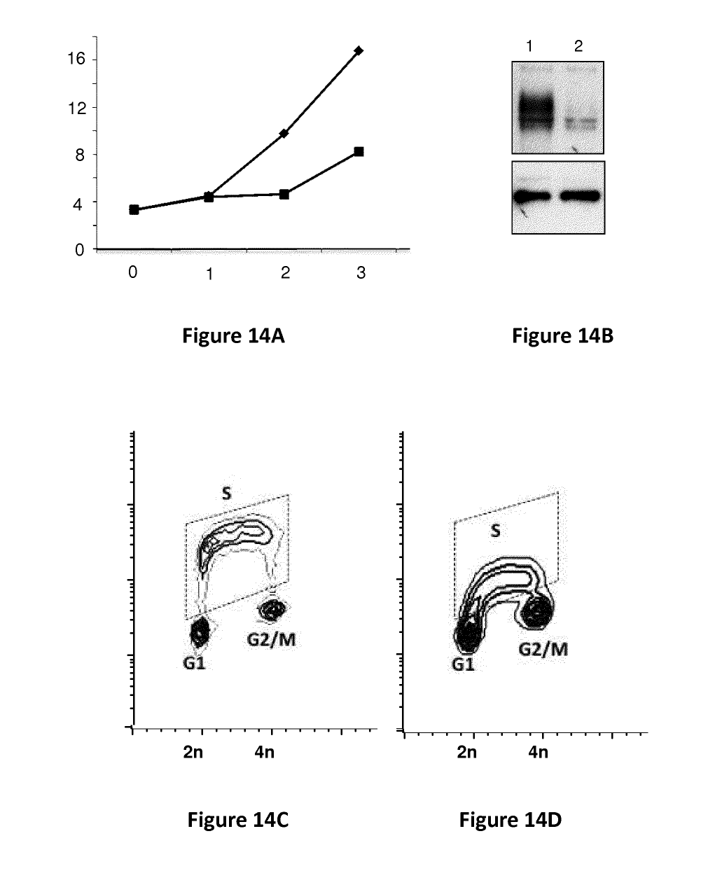

FIG. 14A show that cells transfected with siRNA directed against Obi1 presnt a reduced proliferation.

Moreover, FIGS. 14C and D demonstrate that cells treated with siRNA directed against Obi1 present a large decrease of DNA synthesis, in correlation with the Obi1 interaction with the ORC complex.

### Example 10

### Obi1 is Predominantly Present in Nucleus

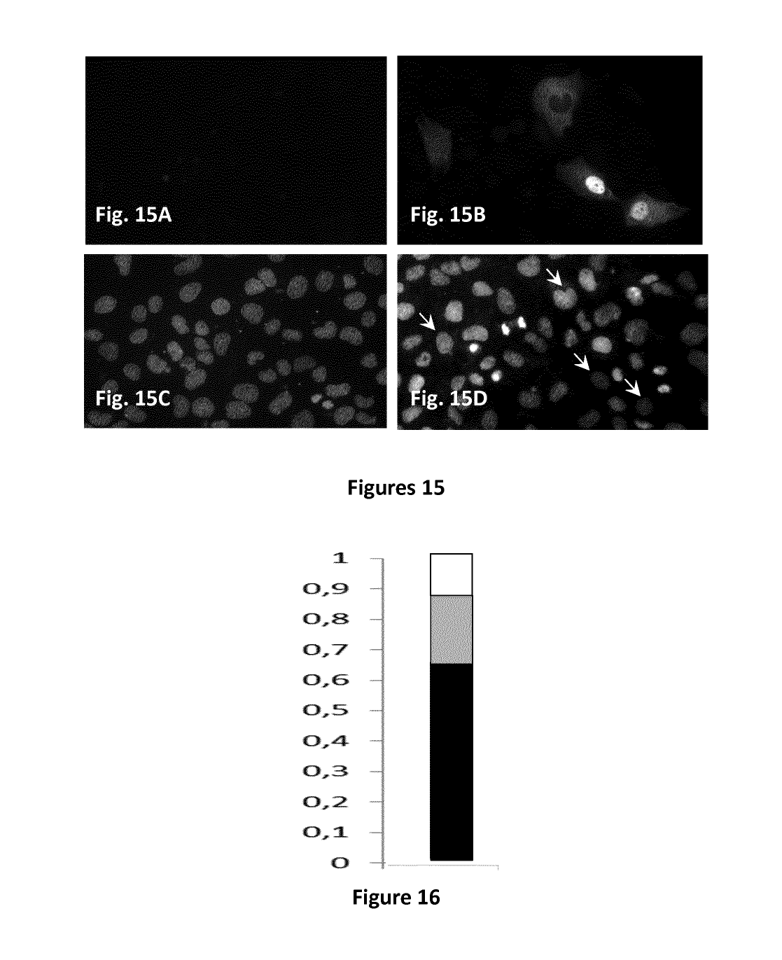

In order to determine where Obi1 protein is expressed, the inventors have transiently expressed a tagged Obi1 protein in U20S cells. Vectors comprising nucleic acid allowing the expression of a Myc-tagged Obi1 protein, or an empty vector were transiently transfected in U20S cells 48 hours post-transfection, cells were fixed in 2% paraformaldehyde/PBS, permabilized and stain using an anti-Myc mouse monoclonal antibody followed by a anti-Mouse FITC coupled antibody. Nuclei were stained using Hoescht (10 mg/ml). (FIGS. 15A-D) Three categories were defined: cells showing a nuclear staining with low cytoplasmic signal (N>C); cells showing an equal distribution between the nuclear and cytosolic compartment (N=C); cells having exclusive cytoplasmic signal (N<C). The inventors noted that Obi1 localisation is predominantly nuclear, with a significant proportion of cells having a strong cytoplasmic distribution (FIG. 16).

### Example 11

### An Artificial Obi1 Protein Lacking the CTD/OBD Domain is a Dominant Negative Protein

To further understand the underlying activity of Obi1 protein during cell cycle, the inventors have developed a mutant form of Obi1 protein. Based on their findings, the inventors know that Obi1 is an homodimeric protein predominantly localized in the nucleus, where it can interact with its substrate, the ORC complex.

The inventors have developed a mutant form of Obi1 lacking the CTD/OBD domain and including a nuclear export sequence (NES), allowing the exit of the protein comprising it from the nucleus.

By fusion of the Obi1 dimerization domain (Coiled-coil) with a Nuclear Export Sequence (NES, derived from PKI), the inventors hypothesised to drive endogenous Obi1 out of the nucleus and away from its substrate. In addition, the heterodimer NES-Obi1(1-280)/Obi1 WT is inactive as it is also unable to bind to the ORC Complex. The nucleic acid molecule coding for said NES-Obi1(1-280) protein consists of the sequence as set forth in SEQ ID NO: 105. The NES-Obi1(1-280) protein consists of the amino acid sequence as set forth in SEQ ID NO: 106.

To obtain the Obi1 inhibitor Myc-NES-Obi1(1-280), a vector harboring the nuclear export sequence (NES) of the Protein Kinase A Inhibitor (PKI) was first constructed. For this, the pCS2+MT (SEQ ID NO: 107) vector was digested with NcoI/EcoRI, which removes the 6^{th} Myc tag and ligated with the following annealed oligos:

NES_PKI_NcoBamEcoS
(SEQ ID NO: 108)
CATGTCTCTGGCTCTCAAACTGGCTGGACTGGACATCGGATCCATGG
TCTTG
NES_PKI_NcoBamEcoAS
(SEQ ID NO: 109)
AATTCAAGACCATGGATCCGATGTCCAGTCCAGCCAGTTTGAGAGCC
AGAGA.

This yields pCS2+MT-NES which expressed Myc-NES tagged proteins. Note that the ligated NcoI site is inactivated and a functional NcoI is reintroducted 3′ of the NES sequence. Then, Obi1 ΔCTD/OBD (which is the same as Obi1(1-280)) was amplified by PCR using the cloned Obi1 cDNA as template with the following template:

Rn_1_NcoS:
(SEQ ID NO: 84)
CTGCCATGGCTCAGACCG
Rn_280XbaAS:
(SEQ ID NO: 87)
CTGTCTAGATTATTTGCCATCTGCAGAAAGTG

These primers introduced NcoI/XbaI sites at both end of the cDNA. Using Pfu polymerase (Promega) as specified by the manufacturer: The following PCR conditions were:

### 1) 2 min at 95° C.

### 2) 30 sec at 95° C.

### 3) 30 sec at 58° C.

4) 2 min/kb extension time at 73° C.
5) repeat step 2-4 25 times

### 6) 5 min at 74° C.

### 7) End, 4° C.

PCR products were purified (Qiaquick, Qiagen) and digested with NcoI/XbaI. The DNA fragments were subcloned into NcoI/XbaI digested pCS2+MT-NES. Restriction digestion and sequencing analysis allowed us to screen for the proper construction.

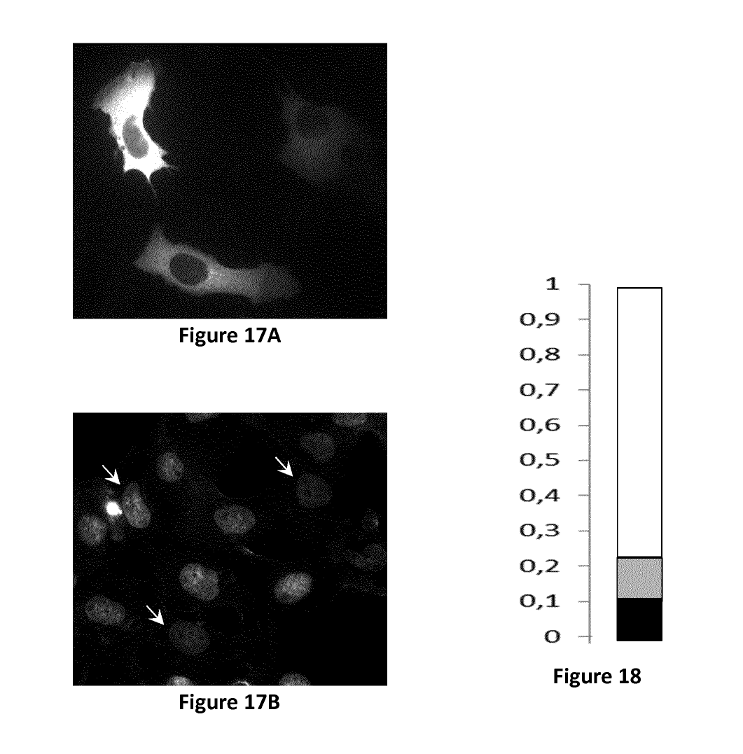

The inventors first assessed the subcellular localisation of the NES-Obi1(1-280) mutant form. U20S cells were transiently transfected with plasmids coding for Myc-tagged NES-Obi1(1-280). 24 hours post-transfection, cells were fixed in 2% paraformaldehyde/PBS, permabilized and stain using an anti-Myc mouse monoclonal antibody followed by a anti-Mouse FITC coupled antibody. Nuclei were stain using Hoescht (10 mg/ml) (FIGS. 17A and 17B). The position of the transfected nuclei is indicated by a white arrow in FIG. 17B. The localization of the ectopic protein was scored on at least 100 cells based on the nuclear:cytoplasm distribution of the signal (FIG. 18). Three categories were defined: cells showing a nuclear staining with low cytoplasmic signal (N>C); cells showing an equal distribution between the nuclear and cytosolic compartment (N=C); cells having exclusive cytoplasmic signal (N<C). Note that Myc-NES-Obi1(1-280) show a strong cytoplasmic signal.

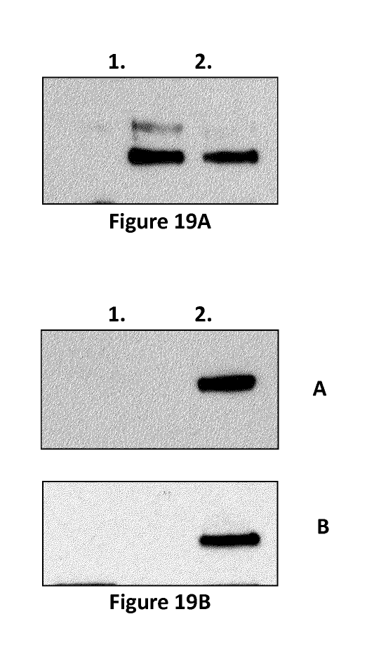

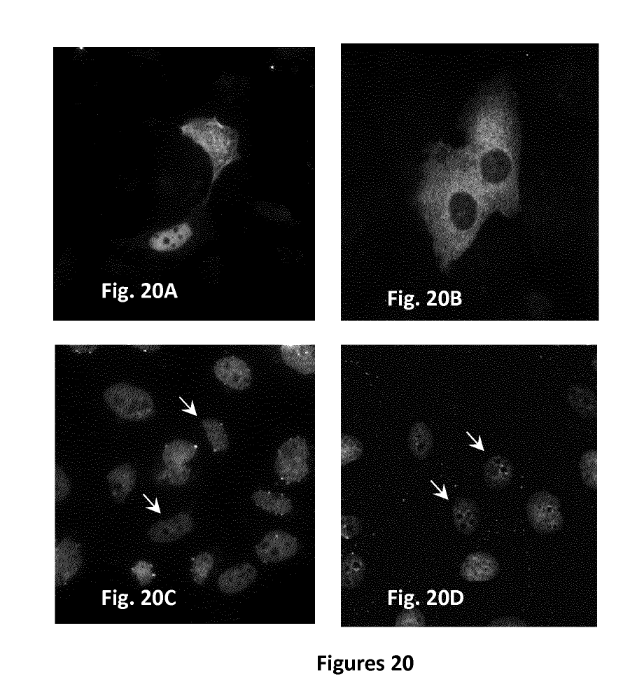

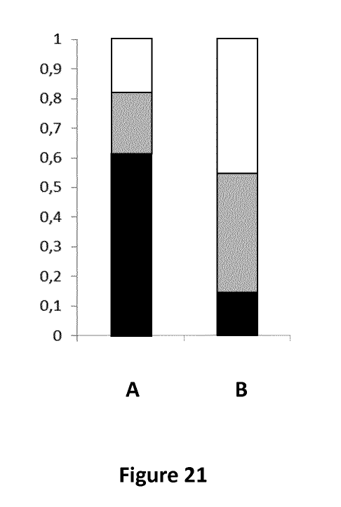

Thus, to confirm that NES-Obi1(1-280) protein still associate with endogenous Obi1 protein, the inventors have tested the interaction between the two proteins. U20S cells were transiently transfected with plasmids coding for Myc-tagged-NES-Obi1(1-280) along with Flag-Obi1 WT. 24 hours post-transfection, cells were lyzed and the soluble fraction (linput, FIG. 19A) was subjected to an anti-Flag immunoprecipitation (IP: Flag). Ectopic proteins were detected by western blotting (FIG. 19B). Under these conditions, specific NES-Obi1(1-280) interaction with WT-Obi1 is detected in the immunoprecipitate. These results show that NES-Obi1(1-280) remains able to associate with the wild type protein.

Further, the inventors have evaluated the cellular localisation of WT-Obi1 protein when co-expressed with NES-Obi1(1-280). U20S cells were transiently transfected with plasmids coding for Flag-tagged WT Obi1 along with Myc-tagged NES-Obi1(1-280) or the empty vector. 24 hours post-transfection, cells were fixed in 2% paraformaldehyde/PBS, permabilized and stain using an anti-Flag mouse monoclonal antibody followed by a anti-Mouse FITC coupled antibody. Nuclei were stain using Hoescht (10 mg/ml). The position of the transfected nuclei is indicated by a white arrow. The localization of the ectopic protein was scored on at least 100 cells based on the nuclear:cytoplasm distribution of the signal. Three categories were defined: cells showing a nuclear staining with low cytoplasmic signal (N>C); cells showing an equal distribution between the nuclear and cytosolic compartment (N=C); cells having exclusive cytoplasmic signal (N<C). These results demonstrate that co-expression of Myc-NES-Obi1(1-280) causes Obi1 to exhibit a stronger cytoplasmic staining, indicating that Myc-NES-Obi1(1-280) acts as a dominant negative form and sequestrate WT-Obi1 protein in the cytoplasm.

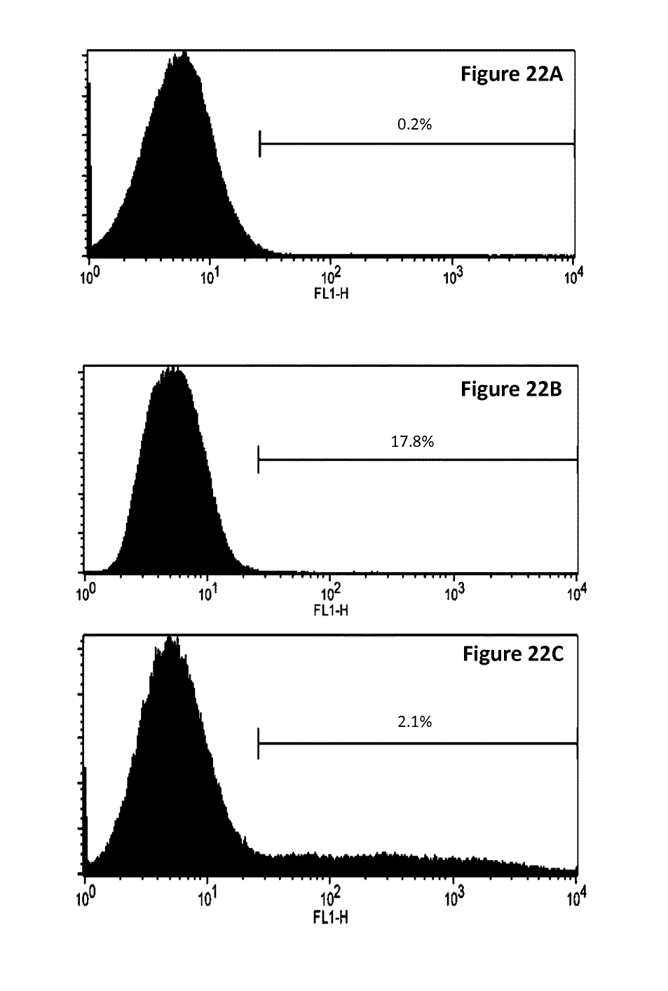

Finally, the inventors evaluated the physiological effect of the Myc-NES-Obi1(1-280) expression. U20S cells were transiently transfected with plasmids coding for EGFP along with Myc-tagged NES-Obi1(1-280) or the empty vector, as indicated. 72 hours post-transfection, cells were trypsined and analyzed for fluorescence by flow cytometry (FIGS. 22A-C). The percentage of EGFP-positive (e.i. transfected) cells is indicated.

These data show that expression of NES-Obi1(1-280) induces a selective loss of transfected cells, possibly by inducing cell cycle arrest or cell death.

These data also strongly suggest that a dominant negative form of Obi1 may influence cell proliferation, and in particular cell proliferation of cancer cells.

### Example 12

### Obi1 Gene is Mutated in Patients Afflicted by Meier-Gorlin Syndrome

To further evaluate the importance of Obi1 gene in regulation of DNA replication, the inventors have evaluated the possible involvement of Obi1 mutation in Meier-Gorlin syndrome patients.

Within a cohort of family having at least one member afflicted by Meier-Gorlin syndrome, the inventors have identified a patient who was heterozygous for a frameshift which lies in the last exon and would predict a truncated protein lacking the last 270 (SEQ ID NO: 100).

The mutation identified by the inventors is C.1370_1371delAA,P.GLU457VALFSX24, i.e. a deletion of the consecutive A at position 1370 and 1371 of the nucleic acid molecule as set forth in SEQ ID NO: 97, resulting in the nucleic acid molecule as set forth in SEQ ID NO: 99. The mutated mRNA expressed in the patient is the nucleic acid molecule as set forth in SEQ ID NO: 98.

The remainder of the gene is covered nicely and there doesn't appear to any second hits. The mother is a carrier for the identified mutation and there is nothing of note in the father.

What is interesting is that in the patient is a query of autosomal dominant inheritance in this family, with the mother being non-penetrant, as apparently the maternal grandmother and the maternal great-grandfather look strikingly similar.

The patient's growth parameters are more mild, like Meier-Gorlin Syndrome, −3.1 Osteitis fibrosa cystica −4.4 height but with osteopenia and a few other bone abnormalities. There is nothing of note in regards to hypomorphic ears or patella.

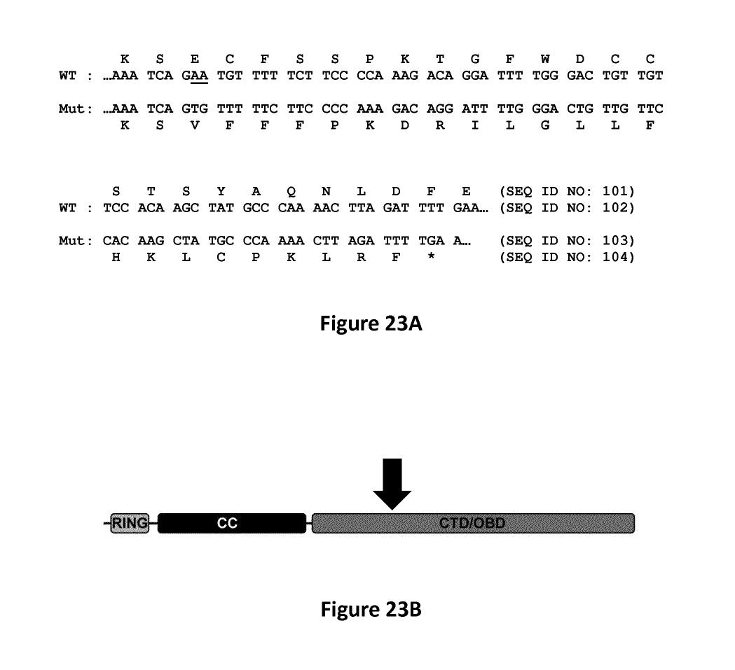

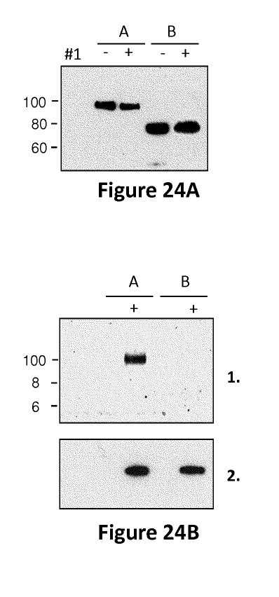

FIG. 23 represents alignment of wild-type Obi1 sequence and mutated (deleted) Obi1 sequence.

### Example 13

### Truncated Obi1 Protein Expressed in Patient Afflicted by Meier-Gorlin Syndrome Fails to Interact with ORC1

In order to characterize the protein expressed by the mutated allele identified in the Meier-Gorlin patient, the inventors have tested its ability to interact with the ORC complex.

U20S cells were transiently transfected with plasmids coding for Myc-tagged WT Obi1 or mutated Obi1, and also co-transfected with with plasmids coding for Flag-ORC1. 48 hours post-transfection, cells were lyzed and the soluble fraction was subjected to an anti-Flag immunoprecipitation (IP:Flag—FIG. 24B). Ectopic proteins were detected by western blotting. Under these conditions, specific Obi1/ORC1 complex is detected in the immunoprecipitate. In contrast, no association is observed between ORC1 and the mutated Obi1 protein (FIG. 24B).

These results demonstrate that truncated Obi1 protein does not interact with ORC, which leads to a modification in DNA replication metabolism.

The natural mutant observed in Meier-Gorlin syndrome appears to act in a similar way than the artificial dominant negative mutant NES-Obi1(1-280) described above, by interfering with the endogenous Obi1 protein activity.

1. A method for treating or preventing a human or animal pathology linked to a dysfunction of the OBI1 gene as set forth in SEQ ID NO: 31 or the Obi1 protein as set forth in SEQ ID NO: 1, or DNA replication, comprising administering to a human or an animal in need thereof a composition comprising at least one compound selected from the group consisting of:
a protein consisting of an amino acid sequence as set forth in SEQ ID NO: 1, or a fragment thereof provided that said fragment retains properties to interact with the origin recognition complex,
a protein derived from the protein as set forth in SEQ ID NO:1 by substitution of at least one nucleotide provided that said protein retains ubiquitin ligase activity and/or participates to DNA replication,
a nucleic acid molecule coding for said proteins or said fragments,
a compound affecting the expression or the activity of said protein, and
a salt or a solvate thereof.

2. The method according to claim 1,
wherein said pathology linked to DNA replication is selected from the group consisting of Meier-Gorlin syndrome, Seckel syndrome and Majewski osteodysplastic primordial dwarfism, and
wherein said composition comprises at least one compound selected from the group consisting of:
a protein consisting of an amino acid sequence as set forth in SEQ ID NO: 1, or a fragment thereof provided that said fragment retains properties to interact with the origin recognition complex,
a protein derived from the protein as set forth in SEQ ID NO:1 by substitution of at least one nucleotide provided that said protein retains ubiquitin ligase activity and/or participates to DNA replication, and
a nucleic acid molecule coding for said proteins or said fragments.

3. The method according to claim 1, wherein said pathology is a neoplastic disease or cancer, and wherein said composition comprises a compound inhibiting the expression of said nucleic acid molecule or the activity of said protein.

4. The method according to claim 1, wherein said compound inhibiting the expression of the protein consisting of an amino acid sequence as set forth in SEQ ID NO: 1, or the protein derived from the protein as set forth in SEQ ID:1 by substitution of at least one nucleotide provided that this protein retains ubiquitin ligase activity and/or participate to DNA replication, is selected from the group consisting of siRNA, miRNA, shRNA, RNA antisense, DNA antisense, antibodies and a fragment of the protein consisting of an amino acid sequence as set forth in SEQ ID NO: 1, said fragment comprising an amino acid sequence selected from the group consisting of SEQ ID NO: 29, SEQ ID NO 30, SEQ ID NO: 59, SEQ ID NO: 60, SEQ ID NO: 61, SEQ ID NO: 62, SEQ ID NO: 63, SEQ ID NO: 100 and SEQ ID NO: 106.

5. The method according to claim 1, wherein said nucleic acid molecule comprises the nucleic acid sequence SEQ ID NO: 31, or a nucleic acid sequence having at least 20% identity with the nucleic acid sequence SEQ ID NO: 31, provided that said nucleic acid molecule code for a protein having ubiquitin ligase activity and/or participates to DNA replication.

6. A nucleic acid molecule inhibiting the expression of the OBI1 gene by RNA interference comprising a sequence selected from the group consisting of SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55, or a combination of said nucleic acid molecule.

7. An inhibitor of the activity of the OBI1 protein, wherein said inhibitor comprises an amino acid sequence selected from the group consisting of: SEQ ID NO: 29, SEQ ID NO 30, SEQ ID NO: 59, SEQ ID NO: 60, SEQ ID NO: 61, SEQ ID NO: 62, SEQ ID NO: 63, SEQ ID NO: 100 and SEQ ID NO: 106.

8. The method according to claim 1, wherein the protein retains activity as a Lys6 ubiquitine ligase enzyme.

9. A method for inhibiting in vitro cell proliferation, comprising administering to a cell an inhibitor of the protein consisting of an amino acid sequence as set forth in SEQ ID NO: 1, or a protein derived from the protein as set forth in SEQ ID:1 by substitution, deletion, or insertion of at least one nucleotide provided that this protein retains ubiquitin ligase activity and/or participate to DNA replication.

10. A process for purifying a protein consisting of the amino acid sequence SEQ ID NO: 1, or a protein derived from the protein as set forth in SEQ ID:1 by substitution, deletion, or insertion of at least one nucleotide provided that this protein retains ubiquitin ligase activity and/or participate to DNA replication, said process comprising:
a step of purifying either the ORC1 protein or the LRWD1 protein, and
a step of isolating the OBI1 protein.

11. An isolated purified protein complex comprising the protein consisting of the amino acid sequence as set forth in SEQ ID NO: 1, or a protein derived from the protein as set forth in SEQ ID:1 by substitution, deletion, or insertion of at least one nucleotide provided that this protein retains ubiquitin ligase activity and/or participate to DNA replication, and the ORC1 protein or the LRWD1 protein, or both.

12. A process for identifying compounds destabilizing the complex defined in claim 11, or inhibiting its activity, said method comprising the steps of:
contacting a cell with a compound liable to destabilize said complex, and
purifying the OBI1 protein as set forth in SEQ ID NO: 1.

13. A method for diagnosing, Meier-Gorlin syndrome, comprising a step of identifying the sequence of the Obi1 gene in cells originating from an individual, and a step of comparing said sequence with a reference sequence as set forth in SEQ ID NO: 97.

14. The method according to claim 1, wherein said at least one compound is in association with a pharmaceutically acceptable carrier.

15. The method according to claim 1, wherein, said nucleic acid molecule is contained in a vector, said vector comprising means allowing the expression of said nucleic acid molecule.
